SEQUENCE LISTING (60 sequences)

SEQ ID NO: 1 - protein, 515 aa, synthetic construct
  REGION 1..515: note = Description of Artificial Sequence: Synthetic polypeptide
EVQLVESGGGLVQAGGSLRLSCAASGRTFSSYAMGWFRQAPGKEREFVVAINWSSGSTYYADSVKGRFTISRDNAKNTMYLQMNSLKPEDTAVYYCAAGYQINSGNYNFKDYEYDYWGQGTQVTVSSGGGGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSHHHHHH

SEQ ID NO: 2 - protein, 515 aa, synthetic construct
  REGION 1..515: note = Description of Artificial Sequence: Synthetic polypeptide
EVQLVESGGGLVQAGGSLRLSCAASGRTFSSYAMGWFRQAPGKEREFVVAINWSSGSTYYADSVKGRFTISRDNAKNTMYLQMNSLKPEDTAVYYCAAGYQINSGNYNFKDYEYDYWGQGTQVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 3 - protein, 515 aa, synthetic construct
  REGION 1..515: note = Description of Artificial Sequence: Synthetic polypeptide
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSGGGSEVQLVESGGGLVQAGGSLRLSCAASGRTFSSYAMGWFRQAPGKEREFVVAINWSSGSTYYADSVKGRFTISRDNAKNTMYLQMNSLKPEDTAVYYCAAGYQINSGNYNFKDYEYDYWGQGTQVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSHHHHHH

SEQ ID NO: 4 - protein, 515 aa, synthetic construct
  REGION 1..515: note = Description of Artificial Sequence: Synthetic polypeptide
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSEVQLVESGGGLVQAGGSLRLSCAASGRTFSSYAMGWFRQAPGKEREFVVAINWSSGSTYYADSVKGRFTISRDNAKNTMYLQMNSLKPEDTAVYYCAAGYQINSGNYNFKDYEYDYWGQGTQVTVSSHHHHHH

SEQ ID NO: 5 - protein, 515 aa, synthetic construct
  REGION 1..515: note = Description of Artificial Sequence: Synthetic polypeptide
EVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSEVQLVESGGGLVQAGGSLRLSCAASGRTFSSYAMGWFRQAPGKEREFVVAINWSSGSTYYADSVKGRFTISRDNAKNTMYLQMNSLKPEDTAVYYCAAGYQINSGNYNFKDYEYDYWGQGTQVTVSSGGGGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 6 - protein, 515 aa, synthetic construct
  REGION 1..515: note = Description of Artificial Sequence: Synthetic polypeptide
EVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSGGGSEVQLVESGGGLVQAGGSLRLSCAASGRTFSSYAMGWFRQAPGKEREFVVAINWSSGSTYYADSVKGRFTISRDNAKNTMYLQMNSLKPEDTAVYYCAAGYQINSGNYNFKDYEYDYWGQGTQVTVSSHHHHHH

SEQ ID NO: 7 - protein, 502 aa, synthetic construct
  REGION 1..502: note = Description of Artificial Sequence: Synthetic polypeptide
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKGGGGSGGGGSGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSASGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 8 - protein, 626 aa, synthetic construct
  REGION 1..626: note = Description of Artificial Sequence: Synthetic polypeptide
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKGGGGSGGGGSGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSASGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSHHHHHH

SEQ ID NO: 9 - protein, 499 aa, synthetic construct
  REGION 1..499: note = Description of Artificial Sequence: Synthetic polypeptide
EVQLVESGGGLVQPGGSLTLSCAASRFMISEYSMHWVRQAPGKGLEWVSTINPAGTTDYAESVKGRFTISRDNAKNTLYLQMNSLKPEDTAVYYCDGYGYRGQGTQVTVSSGGGGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSHHHHHH

SEQ ID NO: 10 - protein, 499 aa, synthetic construct
  REGION 1..499: note = Description of Artificial Sequence: Synthetic polypeptide
EVQLVESGGGLVQPGGSLTLSCAASRFMISEYSMHWVRQAPGKGLEWVSTINPAGTTDYAESVKGRFTISRDNAKNTLYLQMNSLKPEDTAVYYCDGYGYRGQGTQVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 11 - protein, 499 aa, synthetic construct
  REGION 1..499: note = Description of Artificial Sequence: Synthetic polypeptide
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSEVQLVESGGGLVQPGGSLTLSCAASRFMISEYSMHWVRQAPGKGLEWVSTINPAGTTDYAESVKGRFTISRDNAKNTLYLQMNSLKPEDTAVYYCDGYGYRGQGTQVTVSSHHHHHH

SEQ ID NO: 12 - protein, 495 aa, synthetic construct
  REGION 1..495: note = Description of Artificial Sequence: Synthetic polypeptide
EVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSEVQLVESGGGLVQPGGSLTLSCAASRFMISEYSMHWVRQAPGKGLEWVSTINPAGTTDYAESVKGRFTISRDNAKNTLYLQMNSLKPEDTAVYYCDGYGYRGQGTQVTVSSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 13 - protein, 499 aa, synthetic construct
  REGION 1..499: note = Description of Artificial Sequence: Synthetic polypeptide
EVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSGGGSEVQLVESGGGLVQPGGSLTLSCAASRFMISEYSMHWVRQAPGKGLEWVSTINPAGTTDYAESVKGRFTISRDNAKNTLYLQMNSLKPEDTAVYYCDGYGYRGQGTQVTVSSHHHHHH

SEQ ID NO: 14 - protein, 628 aa, synthetic construct
  REGION 1..628: note = Description of Artificial Sequence: Synthetic polypeptide
QVQLVESGGGLVKPGESLRLSCAASGFTFSDYYMYWVRQAPGKGLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYRYSDVPSRFSGSASGTDFTLTISSVQSEDFATYYCQQYDSYPYTFGGGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSHHHHHH

SEQ ID NO: 15 - protein, 504 aa, synthetic construct
  REGION 1..504: note = Description of Artificial Sequence: Synthetic polypeptide
QVQLVESGGGLVKPGESLRLSCAASGFTFSDYYMYWVRQAPGKGLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYRYSDVPSRFSGSASGTDFTLTISSVQSEDFATYYCQQYDSYPYTFGGGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 16 - protein, 451 aa, synthetic construct
  REGION 1..451: note = Description of Artificial Sequence: Synthetic polypeptide
GVTLFVALYDYTSYNTRDLSFHKGEKFQILRMEDGVWWEARSLTTGETGYIPSNYVAPVDSIQGGGGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSHHHHHH

SEQ ID NO: 17 - protein, 500 aa, synthetic construct
  REGION 1..500: note = Description of Artificial Sequence: Synthetic polypeptide
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSQVQLVQSGGGLVQPGGSLRLSCAASDFDFAAYDMSWVRQAPGQGLEWVAIISHDGIDKYYDDSVKGRFTISRDNSKNTLYLQMNTLRAEDTATYQCLRLGAVGQGTLVTVSSHHHHHH

SEQ ID NO: 18 - protein, 500 aa, synthetic construct
  REGION 1..500: note = Description of Artificial Sequence: Synthetic polypeptide
EVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSQVQLVQSGGGLVQPGGSLRLSCAASDFDFAAYDMSWVRQAPGQGLEWVAIISHDGIDKYYDDSVKGRFTISRDNSKNTLYLQMNTLRAEDTATYQCLRLGAVGQGTLVTVSSGGGGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 19 - protein, 500 aa, synthetic construct
  REGION 1..500: note = Description of Artificial Sequence: Synthetic polypeptide
EVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSGGGSQVQLVQSGGGLVQPGGSLRLSCAASDFDFAAYDMSWVRQAPGQGLEWVAIISHDGIDKYYDDSVKGRFTISRDNSKNTLYLQMNTLRAEDTATYQCLRLGAVGQGTLVTVSSHHHHHH

SEQ ID NO: 20 - protein, 243 aa, synthetic construct
  REGION 1..243: note = Description of Artificial Sequence: Synthetic polypeptide
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 21 - protein, 118 aa, synthetic construct [sequence omitted]

SEQ ID NO: 22 - protein, 108 aa, synthetic construct [sequence omitted]

SEQ ID NO: 23 - protein, 119 aa, synthetic construct [sequence omitted]

SEQ ID NO: 24 - protein, 111 aa, synthetic construct
  REGION 1..111: note = Description of Artificial Sequence: Synthetic polypeptide
DIQLTQPNSVSTSLGSTVKLSCTLSSGNIENNYVHWYQLYEGRSPTTMIYDDDKRPDGVPDRFSGSIDRSSNSAFLTIHNVAIEDEAIYFCHSYVSSFNVFGGGTKLTVLR

SEQ ID NO: 25 - protein, 243 aa, synthetic construct
  REGION 1..243: note = Description of Artificial Sequence: Synthetic polypeptide
DVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSSGEGTSTGSGGSGGSGGADDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIK

SEQ ID NO: 26 - protein, 119 aa, synthetic construct [sequence omitted]

SEQ ID NO: 27 - protein, 107 aa, synthetic construct
  REGION 1..107: note = Description of Artificial Sequence: Synthetic polypeptide
DIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITR

SEQ ID NO: 28 - protein, 125 aa, synthetic construct
  REGION 1..125: note = Description of Artificial Sequence: Synthetic polypeptide
EVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSS

SEQ ID NO: 29 - protein, 109 aa, synthetic construct
  REGION 1..109: note = Description of Artificial Sequence: Synthetic polypeptide
QAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVL

SEQ ID NO: 30 - protein, 123 aa, synthetic construct
  REGION 1..123: note = Description of Artificial Sequence: Synthetic polypeptide
EVQLVESGGGLVQPGRSLRLSCAASGFTFDDYTMHWVRQAPGKGLEWVSGISWNSGSIGYADSVKGRFTISRDNAKKSLYLQMNSLRAEDTALYYCAKDNSGYGHYYYGMDVWGQGTTVTVAS

SEQ ID NO: 31 - protein, 108 aa, synthetic construct
  REGION 1..108: note = Description of Artificial Sequence: Synthetic polypeptide
AEIVMTQSPATLSVSPGERATLSCRASQSVSSNLAWYQQKPGQAPRLLIYGASTRATGIPARFSGSGSGTEFTLTISSLQSEDFAVYYCQHYINWPLTFGGGTKVEIK

SEQ ID NO: 32 - protein, 121 aa, synthetic construct
  REGION 1..121: note = Description of Artificial Sequence: Synthetic polypeptide
QVQLQQSGAELARPGASVKMSCKASGYTFTRSTMHWVKQRPGQGLEWIGYINPSSAYTNYNQKFKDKATLTADKSSSTAYMQLSSLTSEDSAVYYCASRQVHYDYNGFPYWGQGTLVTVSS

SEQ ID NO: 33 - protein, 107 aa, synthetic construct
  REGION 1..107: note = Description of Artificial Sequence: Synthetic polypeptide
QVVLTQSPAIMSAFPGEKVTMTCSASSSVSYMNWYQQKSGTSPKRWIYDSSKLASGVPARFSGSGSGTSYSLTISSMETEDAATYYCQQWSRNPPTFGGGTKLQITR

SEQ ID NO: 34 - protein, 125 aa, synthetic construct
  REGION 1..125: note = Description of Artificial Sequence: Synthetic polypeptide
EVKLLESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSA

SEQ ID NO: 35 - protein, 110 aa, synthetic construct
  REGION 1..110: note = Description of Artificial Sequence: Synthetic polypeptide
QAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKLTVLG

SEQ ID NO: 36 - protein, 249 aa, synthetic construct
  REGION 1..249: note = Description of Artificial Sequence: Synthetic polypeptide
EVQLVESGGGLVQPGGSLRLSCAASGFTFNTYAMNWVRQAPGKGLEWVGRIRSKYNNYATYYADSVKGRFTISRDDSKNTLYLQMNSLRAEDTAVYYCVRHGNFGNSYVSWFAYWGQGTLVTVSSGGGGSGGGGSGGGGSQAVVTQEPSLTVSPGGTVTLTCGSSTGAVTTSNYANWVQQKPGQAPRGLIGGTNKRAPGVPARFSGSLLGGKAALTLSGAQPEDEAEYYCALWYSNLWVFGGGTKLTVL

SEQ ID NO: 37 - protein, 245 aa, synthetic construct
  REGION 1..245: note = Description of Artificial Sequence: Synthetic polypeptide
QVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSSAKTTPDIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINRADTAAAGSHHHHHH

SEQ ID NO: 38 - protein, 123 aa, synthetic construct
  REGION 1..123: note = Description of Artificial Sequence: Synthetic polypeptide
EVQLLESGGGLVQPGGSLRLSCAASGFTFSKYWMSWVRQAPGKGLEWVSSIDFMGPHTYYADSVKGRFTISRDNSKNTLYLQMNSLRAEDTAVYYCAKGRTSMLPMKGKFDYWGQGTLVTVSS

SEQ ID NO: 39 - protein, 118 aa, synthetic construct
  REGION 1..118: note = Description of Artificial Sequence: Synthetic polypeptide
EVQLLESGGGLVQPGGSLRLSCTASGFTFDEYNMSWVRQAPGKGLEWVSTILPHGDRTYYADSVKGRFTISRDNSKNTLYLQMNSLRAEDTAVYYCAKQDPLYRFDYWGQGTLVTVSS

SEQ ID NO: 40 - protein, 108 aa, synthetic construct
  REGION 1..108: note = Description of Artificial Sequence: Synthetic polypeptide
DIQMTQSPSSLSASVGDRVTITCRASQKIATYLNWYQQKPGKAPKLLIYRSSSLQSAVPSRFSGSGSGTVFTLTISSLQPEDFATYYCQQTYAVPPTFGQGTKVEIKR

SEQ ID NO: 41 - protein, 108 aa, synthetic construct
  REGION 1..108: note = Description of Artificial Sequence: Synthetic polypeptide
DIQMTQSPSSLSASVGDRVTITCRASQSISSYLNWYQQKPGKAPKLLIYRNSPLQSGVPSRFSGSGSGTDFTLTISSLQPEDFATYYCQQTYRVPPTFGQGTKVEIKR

SEQ ID NO: 42 - protein, 115 aa, synthetic construct
  REGION 1..115: note = Description of Artificial Sequence: Synthetic polypeptide
QVQLQESGGGLVQPGGSLRLSCEASGFTFSRFGMTWVRQAPGKGVEWVSGISSLGDSTLYADSVKGRFTISRDNAKNTLYLQMNSLKPEDTAVYYCTIGGSLNPGGQGTQVTVSS

SEQ ID NO: 43 - protein, 45 aa, synthetic construct
  REGION 1..45: note = Description of Artificial Sequence: Synthetic polypeptide
LKEAKEKAIEELKKAGITSDYYFDLINKAKTVEGVNALKDEILKA

SEQ ID NO: 44 - protein, 121 aa, synthetic construct
  REGION 1..121: note = Description of Artificial Sequence: Synthetic polypeptide
EVQLLESGGGLVQPGGSLRLSCAVSGIDLSNYAINWVRQAPGKCLEWIGIIWASGTTFYATWAKGRFTISRDNSKNTVYLQMNSLRAEDTAVYYCARTVPGYSTAPYFDLWGQGTLVTVSS

SEQ ID NO: 45 - protein, 112 aa, synthetic construct
  REGION 1..112: note = Description of Artificial Sequence: Synthetic polypeptide
DIQMTQSPSSVSASVGDRVTITCQSSPSVWSNFLSWYQQKPGKAPKLLIYEASKLTSGVPSRFSGSGSGTDFTLTISSLQPEDFATYYCGGGYSSISDTTFGCGTKVEIKRT

SEQ ID NO: 46 - protein, 115 aa, synthetic construct [sequence omitted]

SEQ ID NO: 47 - protein, 115 aa, synthetic construct [sequence omitted]

SEQ ID NO: 48 - protein, 117 aa, synthetic construct
  REGION 1..117: note = Description of Artificial Sequence: Synthetic polypeptide
AVQLVESGGGLVQGGGSLRLACAASERIFDLNLMGWYRQGPGNERELVATCITVGDSTNYADSVKGRFTISMDYTKQTVYLHMNSLRPEDTGLYYCKIRRTWHSELWGQGTQVTVSS

SEQ ID NO: 49 - protein, 20 aa, synthetic construct
  REGION 1..20: note = Description of Artificial Sequence: Synthetic peptide
  VARIANT 1..20: note = MISC_FEATURE - This sequence may encompass 1-10 GS repeating units, wherein some positions may be absent
GSGSGSGSGSGSGSGSGSGS

SEQ ID NO: 50 - protein, 30 aa, synthetic construct
  REGION 1..30: note = Description of Artificial Sequence: Synthetic polypeptide
  VARIANT 1..30: note = MISC_FEATURE - This sequence may encompass 1-10 GGS repeating units, wherein some positions may be absent
GGSGGSGGSGGSGGSGGSGGSGGSGGSGGS

SEQ ID NO: 51 - protein, 40 aa, synthetic construct
  REGION 1..40: note = Description of Artificial Sequence: Synthetic polypeptide
  VARIANT 1..40: note = MISC_FEATURE - This sequence may encompass 1-10 GGGS repeating units, wherein some positions may be absent
GGGSGGGSGGGSGGGSGGGSGGGSGGGSGGGSGGGSGGGS

SEQ ID NO: 52 - protein, 40 aa, synthetic construct
  REGION 1..40: note = Description of Artificial Sequence: Synthetic polypeptide
  VARIANT 1..40: note = MISC_FEATURE - This sequence may encompass 1-10 GGSG repeating units, wherein some positions may be absent
GGSGGGSGGGSGGGSGGGSGGGSGGGSGGGSGGGSGGGSG

SEQ ID NO: 53 - protein, 50 aa, synthetic construct
  REGION 1..50: note = Description of Artificial Sequence: Synthetic polypeptide
  VARIANT 1..50: note = MISC_FEATURE - This sequence may encompass 1-10 GGSGG repeating units, wherein some positions may be absent
GGSGGGGSGGGGSGGGGSGGGGSGGGGSGGGGSGGGGSGGGGSGGGGSGG

SEQ ID NO: 54 - protein, 50 aa, synthetic construct
  REGION 1..50: note = Description of Artificial Sequence: Synthetic polypeptide
  VARIANT 1..50: note = MISC_FEATURE - This sequence may encompass 1-10 GGGGS repeating units, wherein some positions may be absent
GGGGSGGGGSGGGGSGGGGSGGGGSGGGGSGGGGSGGGGSGGGGSGGGGS

SEQ ID NO: 55 - protein, 20 aa, synthetic construct
  REGION 1..20: note = Description of Artificial Sequence: Synthetic peptide
GGGGSGGGGSGGGGSGGGGS

SEQ ID NO: 56 - protein, 15 aa, synthetic construct
  REGION 1..15: note = Description of Artificial Sequence: Synthetic peptide
GGGGSGGGGSGGGGS

SEQ ID NO: 57 - protein, 5 aa, unidentified
  REGION 1..5: note = Description of Unknown: Sortase recognition sequence
LPETG

SEQ ID NO: 58 - protein, 5 aa, synthetic construct
  REGION 1..5: note = Description of Artificial Sequence: Synthetic peptide
GGGGS

SEQ ID NO: 59 - protein, 6 aa, synthetic construct
  REGION 1..6: note = Description of Artificial Sequence: Synthetic 6xHis tag
HHHHHH

SEQ ID NO: 60 - protein, 6 aa, unidentified
  REGION 1..6: note = Description of Unknown: Sortase recognition sequence
LPETGG

(19) United States
(12) Patent Application Publication
(10) Pub. No.: US 2025/0197526 A1
(43) Pub. Date: Jun. 19, 2025

(54) TRISPECIFIC BINDING PROTEINS AND METHODS OF USE

(71) Applicant: Harpoon Therapeutics, Inc., South San Francisco, CA (US)

(72) Inventors: Patrick BAEUERLE, Gauting (DE); Luke EVNIN, San Francisco, CA (US); Jeanmarie GUENOT, San Francisco, CA (US); Vanitha RAMAKRISHNAN, Belmont, CA (US); Holger WESCHE, San Francisco, CA (US)

(21) Appl. No.: 18/789,157

(22) Filed: Jul. 30, 2024

Related U.S. Application Data
(63) Continuation of application No. 17/165,760, filed on Feb. 2, 2021, now Pat. No. 12,084,518.
Application No. 17/165,760 is a continuation of application No. 15/630,259, filed on Jun. 22, 2017, now Pat. No. 10,954,311.
Application No. 15/630,259 is a continuation of application No. 15/160,984, filed on May 20, 2016, now Pat. No. 9,708,412.
(60) Provisional application No. 62/305,088, filed on Mar. 8, 2016.
(60) Provisional application No. 62/165,833, filed on May 22, 2015.
(60) Provisional application No. 62/165,153, filed on May 21, 2015.

(51) Int. Cl. C07K 16/46; C07K 16/18; C07K 16/28; C07K 16/30

(52) U.S. Cl. CPC C07K 16/468; C07K 16/18; C07K 16/2809; C07K 16/3069; C07K 2317/31; C07K 2317/569; C07K 2317/622; C07K 2317/92

(57) ABSTRACT

Provided herein are trispecific antigen-binding proteins comprising a domain binding to CD3, a half-life extension domain, and a domain binding to a target antigen. Also provided are pharmaceutical compositions thereof, as well as nucleic acids, recombinant expression vectors and host cells for making such trispecific antigen-binding proteins. Also disclosed are methods of using the disclosed trispecific antigen-binding proteins in the prevention, and/or treatment diseases, conditions and disorders.

### CROSS-REFERENCE

This application is a continuation of U.S. patent application Ser. No. 17/165,760, filed on Feb. 2, 2021, which is a continuation of U.S. patent application Ser. No. 15/630,259, filed Jun. 22, 2017 (now U.S. Pat. No. 10,954,311, issued Mar. 23, 2021); which is a continuation of U.S. patent application Ser. No. 15/160,984, filed May 20, 2016 (now U.S. Pat. No. 9,708,412, issued Jul. 18, 2017); which claims the benefit of U.S. Provisional Application No. 62/305,088, filed Mar. 8, 2016; U.S. Provisional Application No. 62/165,833, filed May 22, 2015; and U.S. Provisional Application No. 62/165,153, filed May 21, 2015, all of which are incorporated herein by reference in their entirety.

### SEQUENCE LISTING

The instant application contains a Sequence Listing which has been submitted electronically in XML format and is hereby incorporated by reference in its entirety. Said XML copy, created on Oct. 10, 2024, is named 25973-US-CNT-3_SL.xml and is 94,129 bytes in size.

### BACKGROUND OF THE INVENTION

The selective destruction of an individual cell or a specific cell type is often desirable in a variety of clinical settings. For example, it is a primary goal of cancer therapy to specifically destroy tumor cells, while leaving healthy cells and tissues intact and undamaged. One such method is by inducing an immune response against the tumor, to make immune effector cells such as natural killer (NK) cells or cytotoxic T lymphocytes (CTLs) attack and destroy tumor cells.

### SUMMARY OF THE INVENTION

Provided herein are trispecific antigen-binding protein, pharmaceutical compositions thereof, as nucleic acids, recombinant expression vectors and host cells for making such trispecific antigen-binding proteins, and methods of use for the treatment of diseases, disorders, or conditions. In one aspect, described herein are trispecific antigen-binding proteins wherein said proteins comprise (a) a first domain (A) which specifically binds to human CD3; (b) a second domain (B) which is a half-life extension domain; and (c) a third domain (C) which specifically binds to a target antigen, wherein the domains are linked in the order H₂N-(A)-(B)-(C)-COOH, H₂N-(A)-(C)-(B)-COOH, H₂N-(B)-(A)-(C)-COOH, H₂N-(B)-(C)-(A)-COOH, H₂N-(C)-(B)-(A)-COOH, or H₂N-(C)-(A)-(B)-COOH by linkers L1 and L2.

Also provided herein in certain aspects are trispecific antigen-binding proteins, wherein said proteins comprise (a) a first domain (A) which specifically binds to human CD3; (b) a second domain (B) which is a half-life extension domain; and (c) a third domain (C) which specifically binds to a target antigen, wherein the domains are linked in the order H₂N-(A)-(C)-(B)-COOH, H₂N-(B)-(A)-(C)-COOH, H₂N-(C)-(B)-(A)-COOH, or by linkers L1 and L2.

Also provided herein in certain aspects are trispecific antigen-binding proteins, wherein said proteins comprise (a) a first domain (A) which specifically binds to human CD3; (b) a second domain (B) which is a half-life extension domain; and (c) a third domain (C) which specifically binds to a target antigen, wherein the domains are linked in the order H₂N-(A)-(B)-(C)-COOH, H₂N-(A)-(C)-(B)-COOH, H₂N-(B)-(A)-(C)-COOH, H₂N-(B)-(C)-(A)-COOH, H₂N-(C)-(B)-(A)-COOH, or H₂N-(C)-(A)-(B)-COOH by linkers L1 and L2, and wherein the first domain binds to human CD3 with a KD of greater than 100 nM.

Also provided herein in certain aspects are trispecific antigen-binding proteins, wherein said proteins comprise (a) a first domain (A) which specifically binds to human CD3; (b) a second domain (B) which is a half-life extension domain; and (c) a third domain (C) which specifically binds to a target antigen, wherein the domains are linked in the order H₂N-(A)-(B)-(C)-COOH, H₂N-(A)-(C)-(B)-COOH, H₂N-(B)-(A)-(C)-COOH, H₂N-(B)-(C)-(A)-COOH, H₂N-(C)-(B)-(A)-COOH, or H₂N-(C)-(A)-(B)-COOH by linkers L1 and L2, and wherein the protein has a molecular weight of less than 55 kDa.

Also provided herein in certain aspects are trispecific antigen-binding proteins, wherein said proteins comprise (a) a first domain (A) which specifically binds to human CD3; (b) a second domain (B) which is a half-life extension domain; and (c) a third domain (c) which specifically binds to a target antigen, wherein the domains are linked in the order H₂N-(A)-(B)-(C)-COOH, H₂N-(A)-(C)-(B)-COOH, H₂N-(B)-(A)-(C)-COOH, H₂N-(B)-(C)-(A)-COOH, H₂N-(C)-(B)-(A)-COOH, or H₂N-(C)-(A)-(B)-COOH by linkers L1 and L2, and wherein B comprises a single domain antibody that binds to serum albumin.

Various embodiments of trispecific antigen-binding proteins are also provided herein, contemplated for any aspect herein, alone or in combination. In some embodiments, first domain comprises a variable light chain and variable heavy chain each of which is capable of specifically binding to human CD3. In some embodiments, the variable light chain is a λ (lambda) light chain. In some embodiments, the variable light chain is a κ (kappa) light chain. In some embodiments, the first domain comprises a single-chain variable fragment (scFv) specific to human CD3. In some embodiments, the first domain is specific for CD3ε (epsilon). In some embodiments, the first domain is specific for CD3δ (delta). In some embodiments, the first domain is specific for CD3γ (gamma). In some embodiments, the first domain comprises complementary determining regions (CDRs) selected from the group consisting of muromonab-CD3 (OKT3), otelixizumab (TRX4), teplizumab (MGA031), visilizumab (Nuvion), SP34, X35, VIT3, BMA030 (BW264/56), CLB-T3/3, CRIS7, YTH12.5, F111-409, CLB-T3.4.2, TR-66, WT32, SPv-T3b, 11D8, XIII-141, XIII-46, XIII-87, 12F6, T3/RW2-8C8, T3/RW2-4B6, OKT3D, M-T301, SMC2, F101.01, UCHT-1 and WT-31. In some embodiments, the first domain is humanized or human. In some embodiments, the first domain has a KD binding of 1000 nM or less to CD3 on CD3 expressing cells. In some embodiments, the first domain has a KD binding of 100 nM or less to CD3 on CD3 expressing cells. In some embodiments, the first domain has a KD binding of 10 nM or less to CD3 on CD3 expressing cells. In some embodiments, the first domain has crossreactivity with cynomolgus CD3. In some embodiments, the first domain comprises an amino acid sequence provided herein.

In some embodiments, the second domain binds human serum albumin. In some embodiments, the second domain comprises a scFv, a variable heavy domain (VH), a variable light domain (VL), a single domain antibody, a peptide, a ligand, or a small molecule. In some embodiments, the second domain comprises a scFv. In some embodiments, the second domain comprises a VH domain. In some embodiments, the second domain comprises a VL domain. In some embodiments, the second domain comprises a single domain antibody. In some embodiments, the second domain comprises a peptide. In some embodiments, the second domain comprises a ligand. In some embodiments, the second domain comprises a small molecule entity.

In some embodiments, the third domain comprises a scFv, a VH domain, a VL domain, a non-Ig domain, a ligand, a knottin, or a small molecule entity that specifically binds to a target antigen. In some embodiments, the third domain is specific to a cell surface molecule. In some embodiments, the third domain is specific to a tumor antigen.

In some embodiments, linkers L1 and L2 are peptide linkers. In some embodiments, linkers L1 and L2 independently consist of about 20 or less amino acid residues. In some embodiments, linkers L1 and L2 are each, independently, selected from (GS)n (SEQ ID NO: 49), (GGS)n (SEQ ID NO: 50), (GGGS)n (SEQ ID NO: 51), (GGSG)n (SEQ ID NO: 52), (GGSGG)n (SEQ ID NO: 53), or (GGGGS)n (SEQ ID NO: 54), wherein n is 1, 2, 3, 4, 5, 6, 7, 8, 9, or 10. In some embodiments, linkers L1 and L2 are each, independently, (GGGGS)4 (SEQ ID NO: 55) or (GGGGS)3 (SEQ ID NO: 56). In some embodiments, linkers L1 and L2 are chemical linkers.

In some embodiments, the first domain is at the N-terminus of the protein. In some embodiments, the second domain is at the N-terminus of the protein. In some embodiments, the third domain is at the N-terminus of the protein. In some embodiments, the first domain is at the C-terminus of the protein. In some embodiments, the second domain is at the C-terminus of the protein. In some embodiments, the third domain is at the C-terminus of the protein.

In some embodiments, the protein is less than about 80 kDa. In some embodiments, the protein is about 50 to about 75 kDa. In some embodiments, the protein is less than about 50 kDa. In some embodiments, the protein is less than about 40 kDa. In some embodiments, the protein is about 20 to about 40 kDa. In some embodiments, the protein has an elimination half-time of at least about 50 hours. In some embodiments, the protein has an elimination half-time of at least about 100 hours. In some embodiments, the protein has increased tissue penetration as compared to an IgG to the same target antigen.

Also provided herein, in another aspect are polynucleotides encoding trispecific antigen-binding proteins according to any one of the above embodiments. In another aspect provided herein are vectors comprising the described polynucleotides. In another aspect, provided herein are host cells transformed with the described vectors.

In yet another aspect, provided herein are pharmaceutical compositions comprising a trispecific antigen-binding protein of any of the above embodiments, a polynucleotide encoding a trispecific antigen-binding protein of any of the above embodiments, a vector comprising the described polynucleotides, or a host cell transformed with a vector of any of the above embodiments and a pharmaceutically acceptable carrier.

Also provided herein, are processes for the production of trispecific antigen-binding proteins according to any of the aspects and embodiments herein, said process comprising culturing a host transformed or transfected with a vector comprising a nucleic acid sequence encoding any trispecific antigen-binding protein herein under conditions allowing the expression of the protein and recovering and purifying the produced protein from the culture.

Also provided herein are methods for the treatment amelioration of a proliferative disease, a tumorous disease, an inflammatory disease, an immunological disorder, an autoimmune disease, an infectious disease, viral disease, allergic reactions, parasitic reactions, graft-versus-host diseases or host-versus-graft diseases comprising the administration of a trispecific antigen-binding protein of any of the above embodiments to a subject in need of such a treatment or amelioration. In some embodiments, the subject is a human. In some embodiments, the method further comprises administration of an agent in combination with the trispecific antigen-binding protein described herein.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is schematic representation of an exemplary trispecific antigen-binding protein where the protein has a constant core element comprising an anti-CD3ε single chain variable fragment (scFv) and an anti-HSA variable heavy chain region; and a variable target binding domain that can be a VH, scFv, a non-Ig binder, or ligand.

FIG. 2 is schematic representation of additional exemplary trispecific antigen-binding proteins constructed for optimal tissue penetration. FIG. 2 left, an exemplary trispecific antigen-binding protein comprising single domain antibody fragments for all its domains. FIG. 2 middle, an exemplary trispecific antigen-binding protein comprising a knottin that binds to a target antigen. FIG. 2 right, an exemplary trispecific antigen-binding protein comprising a natural ligand that binds to a target antigen.

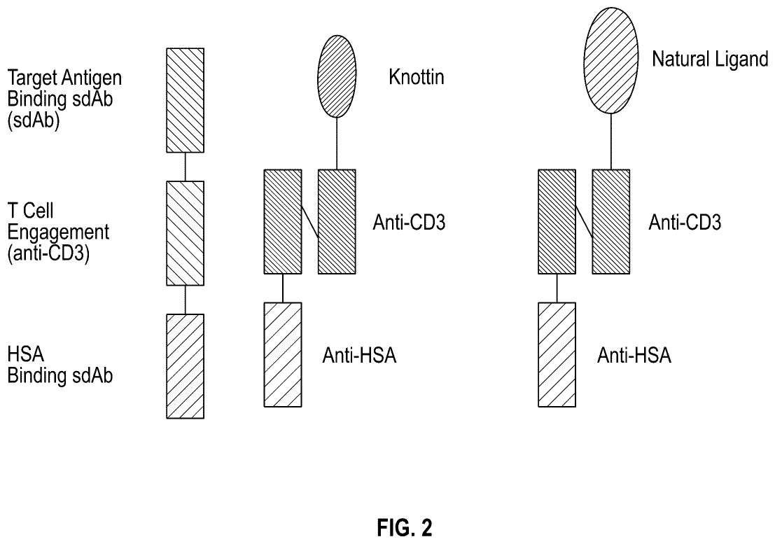

FIG. 3 is a schematic representation of attaching a small molecule entity binder to a trispecific antigen-binding protein. The trispecific antigen-binding protein comprises a sortase recognition sequence as its target antigen binding domain. Upon incubating the protein with a sortase and a glycine-attached small molecule binder, the sortase ligates or conjugates the small molecule binder onto the recognition site. Figure discloses “LPETGG” as SEQ ID NO: 60 and “LPETG” as SEQ ID NO: 57.

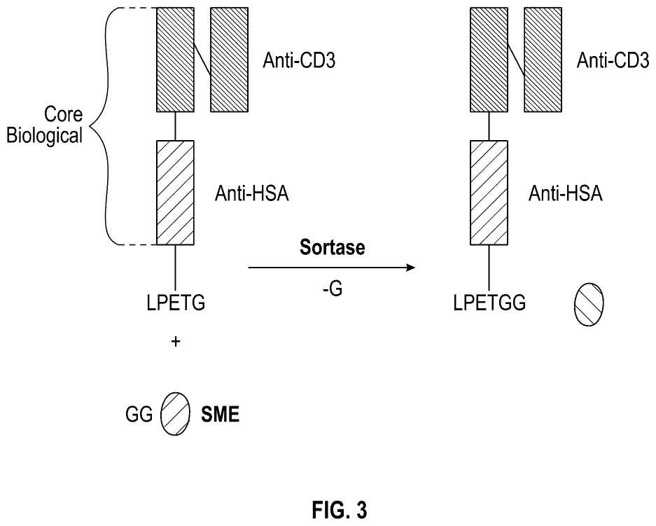

FIG. 4 is schematic representation of the six different ways in which the three domains of these trispecific antigen binding molecules can be arranged.

FIG. 5 compares the ability of BiTE molecules (EGFR targeting BiTE from Lutterbuese et al. 2007. PNAS 107: 12605-12610 and PSMA targeting BiTE pasotuxizumab) with the ability of EGFR and PSMA targeting VH domain containing trispecific molecules to induce primary human T cells to kill tumor cells.

FIG. 6 shows that all six possible configurations of a trispecific molecule containing an EGFR targeting VH domain can induce T cells to kill the human tumor cell line NCI-1563. The experiment was performed in the absence (left side) and presence (right side) of human serum albumin with an EGFR targeting BiTE as positive control.

FIG. 7 assesses the ability of five possible configurations of a trispecific molecule containing a PSMA targeting VH domain to induce T cells to kill the human tumor cell line 22Rv1.The experiment was performed in the absence (left side) and presence (right side) of human serum albumin with a PSMA targeting BiTE as positive control. Also shown is the activity of a PSMA targeting trispecific molecule with a PSMA targeting scFv.

FIG. 8 shows that that the trispecific molecules can consist of a constant core element comprising an anti-CD3ε single chain variable fragment (scFv) and an anti-HSA variable heavy chain region; and a variable target binding domain that can be a scFv.

FIG. 9 demonstrates that trispecific molecules that use a fynomer as opposed to an antibody derived domain for tumor targeting can induce T cells to kill tumor cells.

FIG. 10 shows that when EGFR targeting trispecific molecules redirect T cells to kill human CaPan2 tumor cells (panel A), the T cells get activated and produce the cytokines TNF-α (panel B) and IFNγ (panel C) in a manner dependent on the dose of the trispecific.

FIG. 11 shows that when PSMA targeting trispecific molecules redirect T cells to kill human 22Rv1 tumor cells (panel A), the T cells get activated and produce the cytokines TNF-α (panel B) and IFNγ (panel C) in a manner dependent on the dose of the trispecific.

FIG. 12 shows that MSLN targeting trispecific molecules can migrate through MATRIGEL® faster than conventional antibodies.

FIG. 13 shows phage titration on biotin-CD3ε and biotin-HSA.

### DETAILED DESCRIPTION OF THE INVENTION

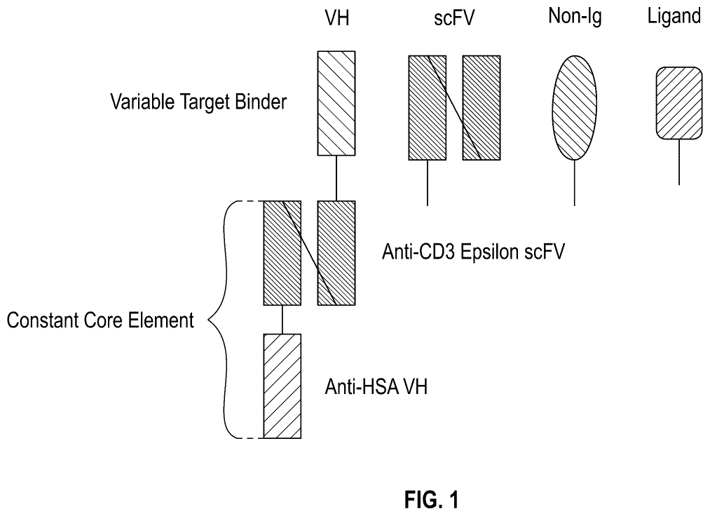

Described herein are trispecific antigen-binding proteins, pharmaceutical compositions thereof, as well as nucleic acids, recombinant expression vectors and host cells for making such trispecific antigen-binding proteins. Also provided are methods of using the disclosed trispecific antigen-binding proteins in the prevention, and/or treatment of diseases, conditions and disorders. The trispecific antigen-binding proteins are capable of specifically binding to a target antigen as well as CD3 and a half-life extension domain, such as a domain binding human serum albumin (HSA). FIG. 1 depicts one non-limiting example of a trispecific antigen-binding protein.

In one aspect, the trispecific antigen-binding proteins comprise a domain (A) which specifically binds to CD3, a domain (B) which specifically binds to human serum albumin (HSA), and a domain (C) which specifically binds to a target antigen. The three domains in trispecific antigen-binding proteins are arranged in any order. Thus, it is contemplated that the domain order of the trispecific antigen-binding proteins are:
H₂N-(A)-(B)-(C)-COOH,
H₂N-(A)-(C)-(B)-COOH,
H₂N-(B)-(A)-(C)-COOH,
H₂N-(B)-(C)-(A)-COOH,
H₂N-(C)-(B)-(A)-COOH, or
H₂N-(C)-(A)-(B)-COOH.

In some embodiments, the trispecific antigen-binding proteins have a domain order of H₂N-(A)-(B)-(C)-COOH. In some embodiments, the trispecific antigen-binding proteins have a domain order of H₂N-(A)-(C)-(B)-COOH. In some embodiments, the trispecific antigen-binding proteins have a domain order of H₂N-(B)-(A)-(C)-COOH. In some embodiments, the trispecific antigen-binding proteins have a domain order of H₂N-(B)-(C)-(A)-COOH. In some embodiments, the trispecific antigen-binding proteins have a domain order of H₂N-(C)-(B)-(A)-COOH. In some embodiments, the trispecific antigen-binding proteins have a domain order of H₂N-(C)-(A)-(B)-COOH.

Trispecific antigen-binding proteins described herein optionally comprise a polypeptide having a sequence described in Table 6 or Table 7 (SEQ ID NOS: 1-48) and subsequences thereof. In some embodiments, the trispecific antigen binding protein comprises a polypeptide having at least 70%-95% or more homology to a sequence described in Table 6 or Table 7 (SEQ ID NOS: 1-48). In some embodiments, the trispecific antigen binding protein comprises a polypeptide having at least 70%, 75%, 80%, 85%, 90%, 95%, or more homology to a sequence described in Table 6 or Table 7 (SEQ ID NO: 1-48). In some embodiments, the trispecific antigen binding protein has a sequence comprising at least a portion of a sequence described in Table 6 or Table 7 (SEQ ID NOS: 1-48). In some embodiments, the trispecific antigen-binding protein comprises a polypeptide comprising one or more of the sequences described in Table 6 or Table 7 (SEQ ID NOS: 1-48).

The trispecific antigen-binding proteins described herein are designed to allow specific targeting of cells expressing a target antigen by recruiting cytotoxic T cells. This improves efficacy compared to ADCC (antibody dependent cell-mediated cytotoxicity), which is using full length antibodies directed to a sole antigen and is not capable of directly recruiting cytotoxic T cells. In contrast, by engaging CD3 molecules expressed specifically on these cells, the trispecific antigen-binding proteins can crosslink cytotoxic T cells with cells expressing a target antigen in a highly specific fashion, thereby directing the cytotoxic potential of the T cell towards the target cell. The trispecific antigen-binding proteins described herein engage cytotoxic T cells via binding to the surface-expressed CD3 proteins, which form part of the TCR. Simultaneous binding of several trispecific antigen-binding protein to CD3 and to a target antigen expressed on the surface of particular cells causes T cell activation and mediates the subsequent lysis of the particular target antigen expressing cell. Thus, trispecific antigen-binding proteins are contemplated to display strong, specific and efficient target cell killing. In some embodiments, the trispecific antigen-binding proteins described herein stimulate target cell killing by cytotoxic T cells to eliminate pathogenic cells (e.g., tumor cells, virally or bacterially infected cells, autoreactive T cells, etc.). In some of such embodiments, cells are eliminated selectively, thereby reducing the potential for toxic side effects. In other embodiments, the same polypeptides could be used to enhance the elimination of endogenous cells for therapeutic effect, such as B or T lymphocytes in autoimmune disease, or hematopoietic stem cells (HSCs) for stem cell transplantation.

The trispecific antigen-binding proteins described herein confer further therapeutic advantages over traditional monoclonal antibodies and other smaller bispecific molecules. Generally, the effectiveness of recombinant protein pharmaceuticals depends heavily on the intrinsic pharmacokinetics of the protein itself. One such benefit here is that the trispecific antigen-binding proteins described herein have extended pharmacokinetic elimination half-time due to having a half-life extension domain such as a domain specific to HSA. In this respect, the trispecific antigen-binding proteins described herein have an extended serum elimination half-time of about two, three, about five, about seven, about 10, about 12, or about 14 days in some embodiments. This contrasts to other binding proteins such as BiTE or DART molecules which have relatively much shorter elimination half-times. For example, the BiTE CD19×CD3 bispecific scFv-scFv fusion molecule requires continuous intravenous infusion (i.v.) drug delivery due to its short elimination half-time. The longer intrinsic half-times of the trispecific antigen-binding proteins solve this issue thereby allowing for increased therapeutic potential such as low-dose pharmaceutical formulations, decreased periodic administration and/or novel pharmaceutical compositions.

The trispecific antigen-binding proteins described herein also have an optimal size for enhanced tissue penetration and tissue distribution. Larger sizes limit or prevent penetration or distribution of the protein in the target tissues. The trispecific antigen-binding proteins described herein avoid this by having a small size that allows enhanced tissue penetration and distribution. Accordingly, the trispecific antigen-binding proteins described herein, In some embodiments, have a size of about 50 kD to about 80 kD, about 50 kD to about 75 kD, about 50 kD to about 70 kD, or about 50 kD to about 65 kD. Thus, the size of the trispecific antigen-binding proteins is advantageous over IgG antibodies which are about 150 kD and the BiTE and DART diabody molecules which are about 55 kD but are not half-life extended and therefore cleared quickly through the kidney.

In further embodiments, the trispecific antigen-binding proteins described herein have an optimal size for enhanced tissue penetration and distribution. In these embodiments, the trispecific antigen-binding proteins are constructed to be as small as possible, while retaining specificity toward its targets. Accordingly, in these embodiments, the trispecific antigen-binding proteins described herein have a size of about 20 kD to about 40 kD or about 25 kD to about 35 kD to about 40 kD, to about 45 kD, to about 50 kD, to about 55 kD, to about 60 kD, to about 65 kD. In some embodiments, the trispecific antigen-binding proteins described herein have a size of about 50kD, 49, kD, 48 kD, 47 kD, 46 kD, 45 kD, 44 kD, 43 kD, 42 kD, 41 kD, 40 kD, about 39 kD, about 38 kD, about 37 kD, about 36 kD, about 35 kD, about 34 kD, about 33 kD, about 32 kD, about 31 kD, about 30 kD, about 29 kD, about 28 kD, about 27 kD, about 26 kD, about 25 kD, about 24 kD, about 23 kD, about 22 kD, about 21 kD, or about 20 kD. An exemplary approach to the small size is through the use of single domain antibody (sdAb) fragments for each of the domains. For example, a particular trispecific antigen-binding protein has an anti-CD3 sdAb, anti-HSA sdAb and an sdAb for a target antigen. This reduces the size of the exemplary trispecific antigen-binding protein to under 40 kD. Thus, in some embodiments, the domains of the trispecific antigen-binding proteins are all single domain antibody (sdAb) fragments. In other embodiments, the trispecific antigen-binding proteins described herein comprise small molecule entity (SME) binders for HSA and/or the target antigen. SME binders are small molecules averaging about 500 to 2000 Da in size and are attached to the trispecific antigen-binding proteins by known methods, such as sortase ligation or conjugation. In these instances, one of the domains of a trispecific antigen-binding protein is a sortase recognition sequence, e.g., LPETG (SEQ ID NO: 57). To attach a SME binder to a trispecific antigen-binding protein with a sortase recognition sequence, the protein is incubated with a sortase and a SME binder whereby the sortase attaches the SME binder to the recognition sequence. Known SME binders include MIP-1072 and MIP-1095 which bind to prostate-specific membrane antigen (PSMA). In yet other embodiments, the domain which binds to a target antigen of a trispecific antigen-binding proteins described herein comprise a knottin peptide for binding a target antigen. Knottins are disulfide-stabilized peptides with a cysteine knot scaffold and have average sizes about 3.5 kD. Knottins have been contemplated for binding to certain tumor molecules such as fibronectin and VEGF-receptor. In further embodiments, domain which binds to a target antigen of a trispecific antigen-binding proteins described herein comprise a natural receptor ligand such as B-cell activating factor (BAFF/BLyS).

Another feature of the trispecific antigen-binding proteins described herein is that they are of a single-polypeptide design with flexible linkage of their domains. This allows for facile production and manufacturing of the trispecific antigen-binding proteins as they can be encoded by single cDNA molecule to be easily incorporated into a vector. Further, because the trispecific antigen-binding proteins described herein are a monomeric single polypeptide chain, there are no chain pairing issues or a requirement for dimerization. It is contemplated that the trispecific antigen-binding proteins described herein have a reduced tendency to aggregate unlike other reported molecules such as bispecific proteins with Fc-gamma immunoglobulin domains.

In the trispecific antigen-binding proteins described herein, the domains are linked by internal linkers L1 and L2, where L1 links the first and second domain of the trispecific antigen-binding proteins and L2 links the second and third domains of the trispecific antigen-binding proteins. Linkers L1 and L2 have an optimized length and/or amino acid composition. In some embodiments, linkers L1 and L2 are the same length and amino acid composition. In other embodiments, L1 and L2 are different. In certain embodiments, internal linkers L1 and/or L2 are “short”, i.e., consist of 0, 1, 2, 3, 4, 5, 6, 7, 8, 9, 10, 11 or 12 amino acid residues. Thus, in certain instances, the internal linkers consist of about 12 or less amino acid residues. In the case of 0 amino acid residues, the internal linker is a peptide bond. In certain embodiments, internal linkers L1 and/or L2 are “long”, i.e., consist of 15, 20 or 25 amino acid residues. In some embodiments, these internal linkers consist of about 3 to about 15, for example 8, 9 or 10 contiguous amino acid residues. Regarding the amino acid composition of the internal linkers L1 and L2, peptides are selected with properties that confer flexibility to the trispecific antigen-binding proteins, do not interfere with the binding domains as well as resist cleavage from proteases. For example, glycine and serine residues generally provide protease resistance. Examples of internal linkers suitable for linking the domains in the trispecific antigen-binding proteins include but are not limited to (GS)ₙ (SEQ ID NO: 49), (GGS)ₙ (SEQ ID NO: 50), (GGGS)ₙ (SEQ ID NO: 51), (GGSG)ₙ (SEQ ID NO: 52), (GGSGG)ₙ (SEQ ID NO: 53), or (GGGGS)ₙ (SEQ ID NO: 54), wherein n is 1, 2, 3, 4, 5, 6, 7, 8, 9, or 10. In one embodiment, internal linker L1 and/or L2 is (GGGGS)₄ (SEQ ID NO: 55) or (GGGGS)₃ (SEQ ID NO: 56).

### CD3 Binding Domain

The specificity of the response of T cells is mediated by the recognition of antigen (displayed in context of a major histocompatibility complex, MHC) by the TCR. As part of the TCR, CD3 is a protein complex that includes a CD3γ (gamma) chain, a CD3δ (delta) chain, and two CD3ε (epsilon) chains which are present on the cell surface. CD3 associates with the α (alpha) and β (beta) chains of the TCR as well as CD3 ζ (zeta) altogether to comprise the complete TCR. Clustering of CD3 on T cells, such as by immobilized anti-CD3 antibodies leads to T cell activation similar to the engagement of the T cell receptor but independent of its clone-typical specificity.

In one aspect, the trispecific antigen-binding proteins described herein comprise a domain which specifically binds to CD3. In one aspect, the trispecific antigen-binding proteins described herein comprise a domain which specifically binds to human CD3. In some embodiments, the trispecific antigen-binding proteins described herein comprise a domain which specifically binds to CD3γ. In some embodiments, the trispecific antigen-binding proteins described herein comprise a domain which specifically binds to CD3δ. In some embodiments, the trispecific antigen-binding proteins described herein comprise a domain which specifically binds to CD3ε.

In further embodiments, the trispecific antigen-binding proteins described herein comprise a domain which specifically binds to the TCR. In certain instances, the trispecific antigen-binding proteins described herein comprise a domain which specifically binds the a chain of the TCR. In certain instances, the trispecific antigen-binding proteins described herein comprise a domain which specifically binds the β chain of the TCR.

In certain embodiments, the CD3 binding domain of the trispecific antigen-binding proteins described herein exhibit not only potent CD3 binding affinities with human CD3 but show also excellent crossreactivity with the respective cynomolgus monkey CD3 proteins. In some instances, the CD3 binding domain of the trispecific antigen-binding proteins are cross-reactive with CD3 from cynomolgus monkey. In certain instances, human: cynomolgous K_{D} ratios for CD3 are between 5 and 0.2.

In some embodiments, the CD3 binding domain of the trispecific antigen-binding protein can be any domain that binds to CD3 including but not limited to domains from a monoclonal antibody, a polyclonal antibody, a recombinant antibody, a human antibody, a humanized antibody. In some instances, it is beneficial for the CD3 binding domain to be derived from the same species in which the trispecific antigen-binding protein will ultimately be used in. For example, for use in humans, it may be beneficial for the CD3 binding domain of the trispecific antigen-binding protein to comprise human or humanized residues from the antigen binding domain of an antibody or antibody fragment.

Thus, in one aspect, the antigen-binding domain comprises a humanized or human antibody or an antibody fragment, or a murine antibody or antibody fragment. In one embodiment, the humanized or human anti-CD3 binding domain comprises one or more (e.g., all three) light chain complementary determining region 1 (LC CDR1), light chain complementary determining region 2 (LC CDR2), and light chain complementary determining region 3 (LC CDR3) of a humanized or human anti-CD3 binding domain described herein, and/or one or more (e.g., all three) heavy chain complementary determining region 1 (HC CDR1), heavy chain complementary determining region 2 (HC CDR2), and heavy chain complementary determining region 3 (HC CDR3) of a humanized or human anti-CD3 binding domain described herein, e.g., a humanized or human anti-CD3 binding domain comprising one or more, e.g., all three, LC CDRs and one or more, e.g., all three, HC CDRs.

In some embodiments, the humanized or human anti-CD3 binding domain comprises a humanized or human light chain variable region specific to CD3 where the light chain variable region specific to CD3 comprises human or non-human light chain CDRs in a human light chain framework region. In certain instances, the light chain framework region is a λ (lambda) light chain framework. In other instances, the light chain framework region is a κ (kappa) light chain framework.

In some embodiments, the humanized or human anti-CD3 binding domain comprises a humanized or human heavy chain variable region specific to CD3 where the heavy chain variable region specific to CD3 comprises human or non-human heavy chain CDRs in a human heavy chain framework region.

In certain instances, the complementary determining regions of the heavy chain and/or the light chain are derived from known anti-CD3 antibodies, such as, for example, muromonab-CD3 (OKT3), otelixizumab (TRX4), teplizumab (MGA031), visilizumab (Nuvion), SP34, TR-66 or X35-3, VIT3, BMA030 (BW264/56), CLB-T3/3, CRIS7, YTH12.5, F111-409, CLB-T3.4.2, TR-66, WT32, SPv-T3b, 11D8, XIII-141, XIII-46, XIII-87, 12F6, T3/RW2-8C8, T3/RW2-4B6, OKT3D, M-T301, SMC2, F101.01, UCHT-1 and WT-31.

In one embodiment, the anti-CD3 binding domain is a single chain variable fragment (scFv) comprising a light chain and a heavy chain of an amino acid sequence provided herein. As used herein, “single chain variable fragment” or “scFv” refers to an antibody fragment comprising a variable region of a light chain and at least one antibody fragment comprising a variable region of a heavy chain, wherein the light and heavy chain variable regions are contiguously linked via a short flexible polypeptide linker, and capable of being expressed as a single polypeptide chain, and wherein the scFv retains the specificity of the intact antibody from which it is derived. In an embodiment, the anti-CD3 binding domain comprises: a light chain variable region comprising an amino acid sequence having at least one, two or three modifications (e.g., substitutions) but not more than 30, 20 or 10 modifications (e.g., substitutions) of an amino acid sequence of a light chain variable region provided herein, or a sequence with 95-99% identity with an amino acid sequence provided herein; and/or a heavy chain variable region comprising an amino acid sequence having at least one, two or three modifications (e.g., substitutions) but not more than 30, 20 or 10 modifications (e.g., substitutions) of an amino acid sequence of a heavy chain variable region provided herein, or a sequence with 95-99% identity to an amino acid sequence provided herein. In one embodiment, the humanized or human anti-CD3 binding domain is a scFv, and a light chain variable region comprising an amino acid sequence described herein, is attached to a heavy chain variable region comprising an amino acid sequence described herein, via a scFv linker. The light chain variable region and heavy chain variable region of a scFv can be, e.g., in any of the following orientations: light chain variable region-scFv linker-heavy chain variable region or heavy chain variable region-scFv linker-light chain variable region.

In some instances, scFvs which bind to CD3 are prepared according to known methods. For example, scFv molecules can be produced by linking VH and VL regions together using flexible polypeptide linkers. The scFv molecules comprise a scFv linker (e.g., a Ser-Gly linker) with an optimized length and/or amino acid composition. Accordingly, in some embodiments, the length of the scFv linker is such that the VH or VL domain can associate intermolecularly with the other variable domain to form the CD3 binding site. In certain embodiments, such scFv linkers are “short”, i.e., consist of 0, 1, 2, 3, 4, 5, 6, 7, 8, 9, 10, 11 or 12 amino acid residues. Thus, in certain instances, the scFv linkers consist of about 12 or less amino acid residues. In the case of 0 amino acid residues, the scFv linker is a peptide bond. In some embodiments, these scFv linkers consist of about 3 to about 15, for example 8, 9 or 10 contiguous amino acid residues. Regarding the amino acid composition of the scFv linkers, peptides are selected that confer flexibility, do not interfere with the variable domains as well as allow inter-chain folding to bring the two variable domains together to form a functional CD3 binding site. For example, scFv linkers comprising glycine and serine residues generally provide protease resistance. In some embodiments, linkers in a scFv comprise glycine and serine residues. The amino acid sequence of the scFv linkers can be optimized, for example, by phage-display methods to improve the CD3 binding and production yield of the scFv. Examples of peptide scFv linkers suitable for linking a variable light chain domain and a variable heavy chain domain in a scFv include but are not limited to (GS)ₙ (SEQ ID NO: 49), (GGS)ₙ (SEQ ID NO: 50), (GGGS)ₙ (SEQ ID NO: 51), (GGSG)ₙ (SEQ ID NO: 52), (GGSGG)ₙ (SEQ ID NO: 53), or (GGGGS)ₙ (SEQ ID NO: 54), wherein n is 1, 2, 3, 4, 5, 6, 7, 8, 9, or 10. In one embodiment, the scFv linker can be (GGGGS)₄ (SEQ ID NO: 55) or (GGGGS)₃ (SEQ ID NO: 56). Variation in the linker length may retain or enhance activity, giving rise to superior efficacy in activity studies.

In some embodiments, CD3 binding domain of a trispecific antigen-binding protein has an affinity to CD3 on CD3 expressing cells with a K_{D} of 1000 nM or less, 500 nM or less, 200 nM or less, 100 nM or less, 80 nM or less, 50 nM or less, 20 nM or less, 10 nM or less, 5 nM or less, 1 nM or less, or 0.5 nM or less. In some embodiments, the CD3 binding domain of a trispecific antigen-binding protein has an affinity to CD3ε, γ, or δ with a K_{D} of 1000 nM or less, 500 nM or less, 200 nM or less, 100 nM or less, 80 nM or less, 50 nM or less, 20 nM or less, 10 nM or less, 5 nM or less, 1 nM or less, or 0.5 nM or less. In further embodiments, CD3 binding domain of a trispecific antigen-binding protein has low affinity to CD3, i.e., about 100 nM or greater.

The affinity to bind to CD3 can be determined, for example, by the ability of the trispecific antigen-binding protein itself or its CD3 binding domain to bind to CD3 coated on an assay plate; displayed on a microbial cell surface; in solution; etc. The binding activity of the trispecific antigen-binding protein itself or its CD3 binding domain of the present disclosure to CD3 can be assayed by immobilizing the ligand (e.g., CD3) or the trispecific antigen-binding protein itself or its CD3 binding domain, to a bead, substrate, cell, etc. Agents can be added in an appropriate buffer and the binding partners incubated for a period of time at a given temperature. After washes to remove unbound material, the bound protein can be released with, for example, SDS, buffers with a high pH, and the like and analyzed, for example, by Surface Plasmon Resonance (SPR).

### Half-Life Extension Domain

Contemplated herein are domains which extend the half-life of an antigen-binding domain. Such domains are contemplated to include but are not limited to HSA binding domains, Fc domains, small molecules, and other half-life extension domains known in the art.

Human serum albumin (HSA) (molecular mass ˜67 kDa) is the most abundant protein in plasma, present at about 50 mg/ml (600 μM) and has a half-life of around 20 days in humans. HSA serves to maintain plasma pH, contributes to colloidal blood pressure, functions as carrier of many metabolites and fatty acids, and serves as a major drug transport protein in plasma.

Noncovalent association with albumin extends the elimination half-time of short-lived proteins. For example, a recombinant fusion of an albumin binding domain to a Fab fragment resulted in an in vivo clearance of 25- and 58-fold and a half-life extension of 26- and 37-fold when administered intravenously to mice and rabbits respectively as compared to the administration of the Fab fragment alone. In another example, when insulin is acylated with fatty acids to promote association with albumin, a protracted effect was observed when injected subcutaneously in rabbits or pigs. Together, these studies demonstrate a linkage between albumin binding and prolonged action.

In one aspect, the trispecific antigen-binding proteins described herein comprise a half-life extension domain, for example a domain which specifically binds to HSA. In some embodiments, the HSA binding domain of a trispecific antigen-binding protein can be any domain that binds to HSA including but not limited to domains from a monoclonal antibody, a polyclonal antibody, a recombinant antibody, a human antibody, a humanized antibody. In some embodiments, the HSA binding domain is a single chain variable fragments (scFv), single-domain antibody such as a heavy chain variable domain (VH), a light chain variable domain (VL) and a variable domain (VHH) of camelid derived single domain antibody, peptide, ligand or small molecule entity specific for HSA. In certain embodiments, the HSA binding domain is a single-domain antibody. In other embodiments, the HSA binding domain is a peptide. In further embodiments, the HSA binding domain is a small molecule. It is contemplated that the HSA binding domain of a trispecific antigen-binding protein is fairly small and no more than 25 kD, no more than 20 kD, no more than 15 kD, or no more than 10 kD in some embodiments. In certain instances, the HSA binding is 5 kD or less if it is a peptide or small molecule entity.

The half-life extension domain of a trispecific antigen-binding protein provides for altered pharmacodynamics and pharmacokinetics of the trispecific antigen-binding protein itself. As above, the half-life extension domain extends the elimination half-time. The half-life extension domain also alters pharmacodynamic properties including alteration of tissue distribution, penetration, and diffusion of the trispecific antigen-binding protein. In some embodiments, the half-life extension domain provides for improved tissue (including tumor) targeting, tissue distribution, tissue penetration, diffusion within the tissue, and enhanced efficacy as compared with a protein without a half-life extension domain. In one embodiment, therapeutic methods effectively and efficiently utilize a reduced amount of the trispecific antigen-binding protein, resulting in reduced side effects, such as reduced non-tumor cell cytotoxicity.

Further, the binding affinity of the half-life extension domain can be selected so as to target a specific elimination half-time in a particular trispecific antigen-binding protein. Thus, in some embodiments, the half-life extension domain has a high binding affinity. In other embodiments, the half-life extension domain has a medium binding affinity. In yet other embodiments, the half-life extension domain has a low or marginal binding affinity. Exemplary binding affinities include K_{D} concentrations at 10 nM or less (high), between 10 nM and 100 nM (medium), and greater than 100 nM (low). As above, binding affinities to HSA are determined by known methods such as Surface Plasmon Resonance (SPR).

### Target Antigen Binding Domain

In addition to the described CD3 and half-life extension domains, the trispecific antigen-binding proteins described herein also comprise a domain that binds to a target antigen. A target antigen is involved in and/or associated with a disease, disorder or condition. In particular, a target antigen associated with a proliferative disease, a tumorous disease, an inflammatory disease, an immunological disorder, an autoimmune disease, an infectious disease, a viral disease, an allergic reaction, a parasitic reaction, a graft-versus-host disease or a host-versus-graft disease. In some embodiments, a target antigen is a tumor antigen expressed on a tumor cell. Alternatively in some embodiments, a target antigen is associated with a pathogen such as a virus or bacterium.

In some embodiments, a target antigen is a cell surface molecule such as a protein, lipid or polysaccharide. In some embodiments, a target antigen is a on a tumor cell, virally infected cell, bacterially infected cell, damaged red blood cell, arterial plaque cell, or fibrotic tissue cell.

The design of the trispecific antigen-binding proteins described herein allows the binding domain to a target antigen to be flexible in that the binding domain to a target antigen can be any type of binding domain, including but not limited to, domains from a monoclonal antibody, a polyclonal antibody, a recombinant antibody, a human antibody, a humanized antibody. In some embodiments, the binding domain to a target antigen is a single chain variable fragments (scFv), single-domain antibody such as a heavy chain variable domain (VH), a light chain variable domain (VL) and a variable domain (VHH) of camelid derived single domain antibody. In other embodiments, the binding domain to a target antigen is a non-Ig binding domain, i.e., antibody mimetic, such as ANTICALINS®, AFFILINS®, AFFIBODY® molecules, AFFIMERS®, affitins, alphabodies, avimers, DARPINS®, fynomers, Kunitz domain peptides, and monobodies. In further embodiments, the binding domain to a target antigen is a ligand or peptide that binds to or associates with a target antigen. In yet further embodiments, the binding domain to a target antigen is a knottin. In yet further embodiments, the binding domain to a target antigen is a small molecular entity.

### Trispecific Protein Modifications

The trispecific antigen-binding proteins described herein encompass derivatives or analogs in which (i) an amino acid is substituted with an amino acid residue that is not one encoded by the genetic code, (ii) the mature polypeptide is fused with another compound such as polyethylene glycol, or (iii) additional amino acids are fused to the protein, such as a leader or secretory sequence or a sequence for purification of the protein.

Typical modifications include, but are not limited to, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent crosslinks, formation of cystine, formation of pyroglutamate, formylation, gamma carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination.

Modifications are made anywhere in trispecific antigen-binding proteins described herein, including the peptide backbone, the amino acid side chains, and the amino or carboxyl termini. Certain common peptide modifications that are useful for modification of trispecific antigen-binding proteins include glycosylation, lipid attachment, sulfation, gamma-carboxylation of glutamic acid residues, hydroxylation, blockage of the amino or carboxyl group in a polypeptide, or both, by a covalent modification, and ADP-ribosylation.

### Polynucleotides Encoding Trispecific Antigen-Binding Proteins

Also provided, in some embodiments, are polynucleotide molecules encoding a trispecific antigen-binding protein described herein. In some embodiments, the polynucleotide molecules are provided as a DNA construct. In other embodiments, the polynucleotide molecules are provided as a messenger RNA transcript.

The polynucleotide molecules are constructed by known methods such as by combining the genes encoding the three binding domains either separated by peptide linkers or, in other embodiments, directly linked by a peptide bond, into a single genetic construct operably linked to a suitable promoter, and optionally a suitable transcription terminator, and expressing it in bacteria or other appropriate expression system such as, for example CHO cells. In the embodiments where the target antigen binding domain is a small molecule, the polynucleotides contain genes encoding the CD3 binding domain and the half-life extension domain. In the embodiments where the half-life extension domain is a small molecule, the polynucleotides contain genes encoding the domains that bind to CD3 and the target antigen. Depending on the vector system and host utilized, any number of suitable transcription and translation elements, including constitutive and inducible promoters, may be used. The promoter is selected such that it drives the expression of the polynucleotide in the respective host cell.

In some embodiments, the polynucleotide is inserted into a vector, preferably an expression vector, which represents a further embodiment. This recombinant vector can be constructed according to known methods. Vectors of particular interest include plasmids, phagemids, phage derivatives, virii (e.g., retroviruses, adenoviruses, adeno-associated viruses, herpes viruses, lentiviruses, and the like), and cosmids.

A variety of expression vector/host systems may be utilized to contain and express the polynucleotide encoding the polypeptide of the described trispecific antigen-binding protein. Examples of expression vectors for expression in E. coli are pSKK (Le Gall et al., J Immunol Methods. (2004) 285(1):111-27) or pcDNA5 (Invitrogen) for expression in mammalian cells.

Thus, the trispecific antigen-binding proteins as described herein, in some embodiments, are produced by introducing a vector encoding the protein as described above into a host cell and culturing said host cell under conditions whereby the protein domains are expressed, may be isolated and, optionally, further purified.

### Pharmaceutical Compositions

Also provided, in some embodiments, are pharmaceutical compositions comprising a trispecific antigen-binding protein described herein, a vector comprising the polynucleotide encoding the polypeptide of the trispecific antigen-binding proteins or a host cell transformed by this vector and at least one pharmaceutically acceptable carrier. The term “pharmaceutically acceptable carrier” includes, but is not limited to, any carrier that does not interfere with the effectiveness of the biological activity of the ingredients and that is not toxic to the patient to whom it is administered. Examples of suitable pharmaceutical carriers are well known in the art and include phosphate buffered saline solutions, water, emulsions, such as oil/water emulsions, various types of wetting agents, sterile solutions etc. Such carriers can be formulated by conventional methods and can be administered to the subject at a suitable dose. Preferably, the compositions are sterile. These compositions may also contain adjuvants such as preservative, emulsifying agents and dispersing agents. Prevention of the action of microorganisms may be ensured by the inclusion of various antibacterial and antifungal agents.

In some embodiments, of the pharmaceutical compositions, the trispecific antigen-binding protein described herein is encapsulated in nanoparticles. In some embodiments, the nanoparticles are fullerenes, liquid crystals, liposome, quantum dots, superparamagnetic nanoparticles, dendrimers, or nanorods. In other embodiments of the pharmaceutical compositions, the trispecific antigen-binding protein is attached to liposomes. In some instances, the trispecific antigen-binding protein are conjugated to the surface of liposomes. In some instances, the trispecific antigen-binding protein are encapsulated within the shell of a liposome. In some instances, the liposome is a cationic liposome.

The trispecific antigen-binding proteins described herein are contemplated for use as a medicament. Administration is effected by different ways, e.g. by intravenous, intraperitoneal, subcutaneous, intramuscular, topical or intradermal administration. In some embodiments, the route of administration depends on the kind of therapy and the kind of compound contained in the pharmaceutical composition. The dosage regimen will be determined by the attending physician and other clinical factors. Dosages for any one patient depends on many factors, including the patient's size, body surface area, age, sex, the particular compound to be administered, time and route of administration, the kind of therapy, general health and other drugs being administered concurrently. An “effective dose” refers to amounts of the active ingredient that are sufficient to affect the course and the severity of the disease, leading to the reduction or remission of such pathology and may be determined using known methods.

### Methods of Treatment

Also provided herein, in some embodiments, are methods and uses for stimulating the immune system of an individual in need thereof comprising administration of a trispecific antigen-binding protein described herein. In some instances, the administration of a trispecific antigen-binding protein described herein induces and/or sustains cytotoxicity towards a cell expressing a target antigen. In some instances, the cell expressing a target antigen is a cancer or tumor cell, a virally infected cell, a bacterially infected cell, an autoreactive T or B cell, damaged red blood cells, arterial plaques, or fibrotic tissue.

Also provided herein are methods and uses for a treatment of a disease, disorder or condition associated with a target antigen comprising administering to an individual in need thereof a trispecific antigen-binding protein described herein. Diseases, disorders or conditions associated with a target antigen include, but are not limited to, viral infection, bacterial infection, auto-immune disease, transplant rejection, atherosclerosis, or fibrosis. In other embodiments, the disease, disorder or condition associated with a target antigen is a proliferative disease, a tumorous disease, an inflammatory disease, an immunological disorder, an autoimmune disease, an infectious disease, a viral disease, an allergic reaction, a parasitic reaction, a graft-versus-host disease or a host-versus-graft disease. In one embodiment, the disease, disorder or condition associated with a target antigen is cancer. In one instance, the cancer is a hematological cancer. In another instance, the cancer is a solid tumor cancer.

As used herein, in some embodiments, “treatment” or “treating” or “treated” refers to therapeutic treatment wherein the object is to slow (lessen) an undesired physiological condition, disorder or disease, or to obtain beneficial or desired clinical results. For the purposes described herein, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment. In other embodiments, “treatment” or “treating” or “treated” refers to prophylactic measures, wherein the object is to delay onset of or reduce severity of an undesired physiological condition, disorder or disease, such as, for example is a person who is predisposed to a disease (e.g., an individual who carries a genetic marker for a disease such as breast cancer).

In some embodiments, of the methods described herein, the trispecific antigen-binding proteins are administered in combination with an agent for treatment of the particular disease, disorder or condition. Agents include but are not limited to, therapies involving antibodies, small molecules (e.g., chemotherapeutics), hormones (steroidal, peptide, and the like), radiotherapies (γ-rays, X-rays, and/or the directed delivery of radioisotopes, microwaves, UV radiation and the like), gene therapies (e.g., antisense, retroviral therapy and the like) and other immunotherapies. In some embodiments, the trispecific antigen-binding proteins are administered in combination with anti-diarrheal agents, anti-emetic agents, analgesics, opioids and/or non-steroidal anti-inflammatory agents. In some embodiments, the trispecific antigen-binding proteins are administered before, during, or after surgery.

### Certain Definitions

As used herein, “elimination half-time” is used in its ordinary sense, as is described in Goodman and Gillman's The Pharmaceutical Basis of Therapeutics 21-25 (Alfred Goodman Gilman, Louis S. Goodman, and Alfred Gilman, eds., 6th ed. 1980). Briefly, the term is meant to encompass a quantitative measure of the time course of drug elimination. The elimination of most drugs is exponential (i.e., follows first-order kinetics), since drug concentrations usually do not approach those required for saturation of the elimination process. The rate of an exponential process may be expressed by its rate constant, k, which expresses the fractional change per unit of time, or by its half-time, t_{1/2} the time required for 50% completion of the process. The units of these two constants are time 1 and time, respectively. A first-order rate constant and the half-time of the reaction are simply related (k×t_{1/2}=0.693) and may be interchanged accordingly. Since first-order elimination kinetics dictates that a constant fraction of drug is lost per unit time, a plot of the log of drug concentration versus time is linear at all times following the initial distribution phase (i.e. after drug absorption and distribution are complete). The half-time for drug elimination can be accurately determined from such a graph.

### EXAMPLES

### Example 1: Construction of an Exemplary Trispecific Antigen-Binding Protein to CD20

Generation of a scFv CD3 Binding Domain

The human CD3ε chain canonical sequence is Uniprot Accession No. P07766. The human CD3γ chain canonical sequence is Uniprot Accession No. P09693. The human CD3δ chain canonical sequence is Uniprot Accession No. P043234. Antibodies against CD3ε, CD3γ or CD3δ are generated via known technologies such as affinity maturation. Where murine anti-CD3 antibodies are used as a starting material, humanization of murine anti-CD3 antibodies is desired for the clinical setting, where the mouse-specific residues may induce a human-anti-mouse antigen (HAMA) response in subjects who receive treatment of a trispecific antigen-binding protein described herein. Humanization is accomplished by grafting CDR regions from murine anti-CD3 antibody onto appropriate human germline acceptor frameworks, optionally including other modifications to CDR and/or framework regions. As provided herein, antibody and antibody fragment residue numbering follows Kabat (Kabat E. A. et al, 1991; Chothia et al, 1987).

Human or humanized anti-CD3 antibodies are therefore used to generate scFv sequences for CD3 binding domains of a trispecific antigen-binding protein. DNA sequences coding for human or humanized VL and VH domains are obtained, and the codons for the constructs are, optionally, optimized for expression in cells from Homo sapiens. The order in which the VL and VH domains appear in the scFv is varied (i.e., VL-VH, or VH-VL orientation), and three copies of the “G4S” (SEQ ID NO: 58) or “G₄S” (SEQ ID NO: 58) subunit (G₄S)₃ (SEQ ID NO: 56) connect the variable domains to create the scFv domain. Anti-CD3 scFv plasmid constructs can have optional Flag, His or other affinity tags, and are electroporated into HEK293 or other suitable human or mammalian cell lines and purified. Validation assays include binding analysis by FACS, kinetic analysis using PROTEON®, and staining of CD3-expressing cells.

Generation of a scFv CD20 Binding Domain

CD20 is one of the cell surface proteins present on B-lymphocytes. CD20 antigen is found in normal and malignant pre-B and mature B lymphocytes, including those in over 90% of B-cell non-Hodgkin's lymphomas (NHL). The antigen is absent in hematopoietic stem cells, activated B lymphocytes (plasma cells) and normal tissue. As such, several antibodies mostly of murine origin have been described: 1F5, 2B8/C2B8, 2H7, and 1H4.

A scFv binding domain to CD20 is generated similarly to the above method for generation of a scFv binding domain to CD3.

### Cloning of DNA Expression Constructs Encoding the Trispecific Antigen-Binding Protein

The anti-CD3 scFv domains are used to construct a trispecific antigen-binding protein in combination with an anti-CD20 scFv domain and a HSA binding domain (e.g., a peptide or VH domain), with the domains organized as shown FIG. 1. For expression of a trispecific antigen-binding protein in CHO cells, coding sequences of all protein domains are cloned into a mammalian expression vector system. In brief, gene sequences encoding the CD3 binding domain, HSA binding domain, and CD20 binding domain along with peptide linkers L1 and L2 are separately synthesized and subcloned. The resulting constructs are then ligated together in the order of ‘CD20 binding domain—L1—CD3 binding domain—L2—HSA binding domain’ to yield a final construct. All expression constructs are designed to contain coding sequences for an N-terminal signal peptide and a C-terminal hexahistidine (6×His)-tag (SEQ ID NO: 59) to facilitate protein secretion and purification, respectively.

### Expression of Trispecific Antigen-Binding Proteins in Stably Transfected CHO Cells

A CHO cell expression system (FLP-IN®, Life Technologies), a derivative of CHO-K1 Chinese Hamster ovary cells (ATCC, CCL-61) (Kao and Puck, Proc. Natl. Acad Sci USA 1968; 60(4):1275-81), is used. Adherent cells are sub-cultured according to standard cell culture protocols provided by Life Technologies.

For adaption to growth in suspension, cells are detached from tissue culture flasks and placed in serum-free medium. Suspension-adapted cells are cryopreserved in medium with 10% DMSO.

Recombinant CHO cell lines stably expressing secreted trispecific antigen-binding proteins are generated by transfection of suspension-adapted cells. During selection with the antibiotic Hygromycin B viable cell densities are measured twice a week, and cells are centrifuged and resuspended in fresh selection medium at a maximal density of 0.1×10⁶ viable cells/mL. Cell pools stably expressing trispecific antigen-binding proteins are recovered after 2-3 weeks of selection at which point cells are transferred to standard culture medium in shake flasks. Expression of recombinant secreted proteins is confirmed by performing protein gel electrophoresis or flow cytometry. Stable cell pools are cryopreserved in DMSO containing medium.

Trispecific antigen-binding proteins are produced in 10-day fed-batch cultures of stably transfected CHO cell lines by secretion into the cell culture supernatant. Cell culture supernatants are harvested after 10 days at culture viabilities of typically >75%. Samples are collected from the production cultures every other day and cell density and viability are assessed. On day of harvest, cell culture supernatants are cleared by centrifugation and vacuum filtration before further use.

Protein expression titers and product integrity in cell culture supernatants are analyzed by SDS-PAGE.

### Purification of Trispecific Antigen-Binding Proteins

Trispecific antigen-binding proteins are purified from CHO cell culture supernatants in a two-step procedure. The constructs are subjected to affinity chromatography in a first step followed by preparative size exclusion chromatography (SEC) on SUPERDEX® 200 in a second step. Samples are buffer-exchanged and concentrated by ultrafiltration to a typical concentration of >1 mg/mL. Purity and homogeneity (typically >90%) of final samples are assessed by SDS PAGE under reducing and non-reducing conditions, followed by immunoblotting using an anti-HSA or anti idiotype antibody as well as by analytical SEC, respectively. Purified proteins are stored at aliquots at −80° C. until use.

### Example 2: Determination of Antigen Affinity by Flow Cytometry

The trispecific antigen-binding proteins of Example 1 are tested for their binding affinities to human CD3⁺ and CD20⁺ cells and cynomolgus CD3⁺ and CD20⁺ cells.

CD3⁺ and CD20⁺ cells are incubated with 100 μL of serial dilutions of the trispecific antigen-binding proteins of Example 1. After washing three times with FACS buffer the cells are incubated with 0.1 mL of 10 μg/mL mouse monoclonal anti-idiotype antibody in the same buffer for 45 min on ice. After a second washing cycle, the cells are incubated with 0.1 mL of 15 μg/mL FITC-conjugated goat anti-mouse IgG antibodies under the same conditions as before. As a control, cells are incubated with the anti-His IgG followed by the FITC-conjugated goat anti-mouse IgG antibodies without the trispecific antigen-binding proteins. The cells were then washed again and resuspended in 0.2 mL of FACS buffer containing 2 μg/mL propidium iodide (PI) in order to exclude dead cells. The fluorescence of 1×10⁴ living cells is measured using a Beckman-Coulter FC500 MPL flow cytometer using the MXP software (Beckman-Coulter, Krefeld, Germany) or a Millipore Guava EasyCyte flow cytometer using the Incyte software (Merck Millipore, Schwalbach, Germany). Mean fluorescence intensities of the cell samples are calculated using CXP software (Beckman-Coulter, Krefeld, Germany) or Incyte software (Merck Millipore, Schwalbach, Germany). After subtracting the fluorescence intensity values of the cells stained with the secondary and tertiary reagents alone the values are them used for calculation of the K_{D} values with the equation for one-site binding (hyperbola) of the GraphPad Prism (version 6.00 for Windows, GraphPad Software, La Jolla California USA).

CD3 binding affinity and crossreactivity are evaluated in titration and flow cytometric experiments on CD3⁺ Jurkat cells and the cynomolgus CD3⁺ HSC-F cell line (JCRB, cat.: JCRB1164). CD20 binding and crossreactivity are assessed on the human CD20⁺ tumor cell lines. The K_{D} ratio of crossreactivity is calculated using the K_{D} values determined on the CHO cell lines expressing either recombinant human or recombinant cynomolgus antigens.

### Example 3: Cytotoxicity Assay

The trispecific antigen-binding protein of Example 1 is evaluated in vitro on its mediation of T cell dependent cytotoxicity to CD20⁺ target cells.

Fluorescence labeled CD20⁺ REC-1 cells (a Mantle cell lymphoma cell line, ATCC CRL-3004) are incubated with isolated PBMC of random donors or CB15 T-cells (standardized T-cell line) as effector cells in the presence of the trispecific antigen-binding protein of Example 1. After incubation for 4 h at 37° C. in a humidified incubator, the release of the fluorescent dye from the target cells into the supernatant is determined in a spectrofluorometer. Target cells incubated without the trispecific antigen-binding protein of Example 1 and target cells totally lysed by the addition of saponin at the end of the incubation serve as negative and positive controls, respectively.

Based on the measured remaining living target cells, the percentage of specific cell lysis is calculated according to the following formula: [1−(number of living targets₍ₛₐₘₚₗₑ₎/number of living targets₍ₛₚₒₙₜₐₙₑₒᵤₛ₎)]×100%. Sigmoidal dose response curves and EC₅₀ values are calculated by non-linear regression/4-parameter logistic fit using the GraphPad Software. The lysis values obtained for a given antibody concentration are used to calculate sigmoidal dose-response curves by 4 parameter logistic fit analysis using the Prism software.

### Example 4: Pharmacokinetics of Trispecific Antigen-Binding Proteins

The trispecific antigen-binding protein of Example 1 is evaluated for half-time elimination in animal studies.

The trispecific antigen-binding protein is administered to cynomolgus monkeys as a 0.5 mg/kg bolus injection intramuscularly. Another cynomolgus monkey group receives a comparable protein in size with binding domains to CD3 and CD20, but lacking HSA binding. A third and fourth group receive a protein with CD3 and HSA binding domains and a protein with CD20 and HSA binding domains respectively, and both comparable in size to the trispecific antigen-binding protein. Each test group consists of 5 monkeys. Serum samples are taken at indicated time points, serially diluted, and the concentration of the proteins is determined using a binding ELISA to CD3 and/or CD20.

Pharmacokinetic analysis is performed using the test article plasma concentrations. Group mean plasma data for each test article conforms to a multi-exponential profile when plotted against the time post-dosing. The data are fit by a standard two-compartment model with bolus input and first-order rate constants for distribution and elimination phases. The general equation for the best fit of the data for i.v. administration is: c(t)=Ae^{-αt}+Be^{-βt}, where c(t) is the plasma concentration at time t, A and B are intercepts on the Y-axis, and α and β are the apparent first-order rate constants for the distribution and elimination phases, respectively. The α-phase is the initial phase of the clearance and reflects distribution of the protein into all extracellular fluid of the animal, whereas the second or β-phase portion of the decay curve represents true plasma clearance. Methods for fitting such equations are well known in the art. For example, A=D/V(α−k21)/(α−β), B=D/V(β−k21)/(α−β), and α and β (for α>β) are roots of the quadratic equation: r²+(k12+k21+k10)r+k21k10=0 using estimated parameters of V=volume of distribution, k10-elimination rate, k12=transfer rate from compartment 1 to compartment 2 and k21=transfer rate from compartment 2 to compartment 1, and D=the administered dose.

Data analysis: Graphs of concentration versus time profiles are made using KaleidaGraph (KALEIDAGRAPH™ V. 3.09 Copyright 1986-1997. Synergy Software. Reading, Pa.). Values reported as less than reportable (LTR) are not included in the PK analysis and are not represented graphically. Pharmacokinetic parameters are determined by compartmental analysis using WinNonlin software (WINNONLIN® Professional V. 3.1 WINNONLIN™ Copyright 1998-1999. Pharsight Corporation. Mountain View, Calif.). Pharmacokinetic parameters are computed as described in Ritschel W A and Kearns G L, 1999, IN: Handbook of Basic Pharmacokinetics Including Clinical Applications, 5th edition, American Pharmaceutical Assoc., Washington, D.C.

It is expected that the trispecific antigen-binding protein of Example 1 has improved pharmacokinetic parameters such as an increase in elimination half-time as compared to proteins lacking an HSA binding domain.

### Example 5: Xenograft Tumor Model

The trispecific antigen-binding protein of Example 1 is evaluated in a xenograft model.

Female immune-deficient NOD/SCID mice are sub-lethally irradiated (2 Gy) and subcutaneously inoculated with 4×10⁶ Ramos RA1 cells into their the right dorsal flank. When tumors reach 100 to 200 mm³, animals are allocated into 3 treatment groups. Groups 2 and 3 (8 animals each) are intraperitoneally injected with 1.5×10⁷ activated human T-cells. Three days later, animals from Group 3 are subsequently treated with a total of 9 intravenous doses of 50 μg trispecific antigen-binding protein of Example 1 (qdx9d). Groups 1 and 2 are only treated with vehicle. Body weight and tumor volume are determined for 30 days.

It is expected that animals treated with the trispecific antigen-binding protein of Example 1 have a statistically significant delay in tumor growth in comparison to the respective vehicle-treated control group.

### Example 6: Proof-of-Concept Clinical Trial Protocol for Administration of the Trispecific Antigen-Binding Protein of Example 1 to B-Cell Lymphoma Patients

This is a Phase I/II clinical trial for studying the trispecific antigen-binding protein of Example 1 as a treatment for with B-cell Lymphoma.

### Study Outcomes:

Primary: Maximum tolerated dose of trispecific antigen-binding protein of Example 1

Secondary: To determine whether in vitro response of trispecific antigen-binding protein of Example 1 is associated with clinical response

### Phase I

The maximum tolerated dose (MTD) will be determined in the phase I section of the trial.
1.1 The maximum tolerated dose (MTD) will be determined in the phase I section of the trial.
1.2 Patients who fulfill eligibility criteria will be entered into the trial to trispecific antigen-binding protein of Example 1.
1.3 The goal is to identify the highest dose of trispecific antigen-binding protein of Example 1 that can be administered safely without severe or unmanageable side effects in participants. The dose given will depend on the number of participants who have been enrolled in the study prior and how well the dose was tolerated. Not all participants will receive the same dose.

### Phase II

2.1 A subsequent phase II section will be treated at the MTD with a goal of determining if therapy with therapy of trispecific antigen-binding protein of Example 1 results in at least a 20% response rate.
   Primary Outcome for the Phase II—To determine if therapy of trispecific antigen-binding protein of Example 1 results in at least 20% of patients achieving a clinical response (blast response, minor response, partial response, or complete response)

### Eligibility

Histologically confirmed newly diagnosed aggressive B-cell lymphoma according to the current World Health Organisation Classification, from 2001 to 2007
Any stage of disease.
Treatment with R-CHOP or R-CHOP like regimens (+/− transplant).
Age ≥18 years
Karnofsky performance status ≥50% or ECOG performance status 0-2
Life expectancy ≥6 weeks

### Example 7: Methods to Assess Binding and Cytotoxic Activities of Trispecific Antigen Binding Molecules

### Protein Production

Sequences of trispecific molecules were cloned into mammalian expression vector pCDNA 3.4 (Invitrogen) preceded by a leader sequence and followed by a 6× Histidine Tag (SEQ ID NO: 59). Expi293F cells (Life Technologies A14527) were maintained in suspension in Optimum Growth Flasks (Thomson) between 0.2 to 8×1e6 cells/ml in Expi293 media. Purified plasmid DNA was transfected into Expi293 cells in accordance with Expi293 Expression System Kit (Life Technologies, A14635) protocols, and maintained for 4-6 days post transfection. Conditioned media was partially purified by affinity and desalting chromatography. Trispecific proteins were subsequently polished by ion exchange or, alternatively, concentrated with AMICON® Ultra centrifugal filtration units (EMD Millipore), applied to SUPERDEX® 200 size exclusion media (GE Healthcare) and resolved in a neutral buffer containing excipients. Fraction pooling and final purity were assessed by SDS-PAGE and analytical SEC.

### Affinity Measurements

The affinities of the binding domains molecules were measured by biolayer inferometry using an Octet instrument.

PSMA affinities were measured by loading human PSMA-Fc protein (100 nM) onto anti-human IgG Fc biosensors for 120 seconds, followed by a 60 second baseline, after which associations were measured by incubating the sensor tip in a dilution series of the trispecific molecules for 180 seconds, followed by dissociation for 50 seconds. EGFR and CD3 affinities were measured by loading human EGFR-Fc protein or human CD3-Flag-Fc protein, respectively, (100 nM) onto anti-human IgG Fc biosensors for 120 seconds, followed by a 60 second baseline, after which associations were measured by incubating the sensor tip in a dilution series of the trispecific molecules for 180 seconds, followed by dissociation for 300 seconds. Affinities to human serum albumin (HSA) were measured by loading biotinylated albumin onto streptavidin biosensors, then following the same kinetic parameters as for CD3 affinity measurements. All steps were performed at 30° C. in 0.25% casein in phosphate-buffered saline.

### Cytotoxicity Assays

A human T-cell dependent cellular cytotoxicity (TDCC) assay is used to measure the ability of T cell engagers, including trispecific molecules, to direct T cells to kill tumor cells (Nazarian et al. 2015. J Biomol Screen. 20:519-27). In this assay, T cells and target cancer cell line cells are mixed together at a 10:1 ratio in a 384 wells plate, and varying amounts of T cell engager are added. After 48 hours, the T cells are washed away leaving attached to the plate target cells that were not killed by the T cells. To quantitate the remaining viable cells, CELLTITER-GLO® Luminescent Cell Viability Assay (Promega) is used.

### Cytokine Assays

ALPHALISA™ assays (Perkin Elmer) for TNF-alpha and Interferon gamma are used to obtain evidence that T cells are activated by trispecific molecules in the presence of target cells. For this assay, primary human T cells and human tumor cells are incubated in the presence of test molecules as described under cytotoxicity assays. After 48 h of incubation, 2 microliter aliquots of the assay supernatants are analyzed according to the manufacturer's instructions.

### Diffusion Assays

A layer of Matrigel (75 μL) was added to 24 well Transwell inserts (0.4 μm), after which PBS was added to the upper and lower chambers (100 and 1025 μL, respectively) and equilibrated overnight at 4° C. 100 pmol of IgG or Fab (goat anti-human Fc, Jackson ImmunoResearch) or trispecific molecules was added to the upper chamber, and diffusion of each molecule into the lower chamber was quantified over time by an ELISA specific to each molecule. IgG and Fab were captured by donkey anti-goat IgG (Jackson ImmunoResearch) that had been immobilized on ELISA plates and were detected with a horseradish peroxidase conjugated donkey anti-goat IgG (Jackson ImmunoResearch) and TMB development. Trispecific molecules were captured by human serum albumin (Athens Research & Technology) that had been immobilized on ELISA plates, and were detected with a horseradish peroxidase conjugated anti-His antibody (Genscript) and TMB development.

Relative diffusion at each timepoint was calculated as: (concentration in the lower chamber at time=t)/(concentration in the upper chamber at time=t).

Statistically significant differences in diffusion between the IgG molecule and the Fab or trispecific molecules were identified using an unpaired t-test.

### Example 8: Affinity Measurements for EGFR Targeting Trispecific Molecules

The affinities of the three binding domains in the EGFR targeting molecule were measured by biolayer inferometry using an Octet instrument and are summarized in Table 1.

Trispecific molecules in which the EGFR binding domain is located at the N-terminus of the molecule showed significantly higher affinities to EGFR, compared to trispecific molecules that contained the EGFR binding domain in the center or in the C-terminal position. Similarly, the trispecific molecules containing the albumin binding domain at the N-terminus also exhibited higher affinities to HSA than those containing albumin in the middle or C-terminal positions. In contrast, all trispecific molecules exhibited very similar affinities to human CD3, independent of the position of the binding domain within the trispecific molecule.

### Example 9: Affinity Measurements for PSMA Targeting Trispecific Molecules

The affinities of the three binding domains in the PSMA targeting molecules were measured by biolayer inferometry using an Octet instrument and are summarized in Table 2.

Trispecific molecules containing the albumin binding domain at the N-terminus had higher affinities to HSA than those containing the albumin binding domain in the middle or C-terminal positions. In contrast, the position of the CD3 binding domain did not affect the affinity for its target. Likewise, the position of the PSMA binding domain had little impact on affinity, with all trispecific molecules having affinities for human PSMA within 3-fold of each other.

### Example 10: Cytotoxicity Assays with Trispecific Molecules

Trispecific molecules were tested in T cell dependent cytotoxicity (TDCC) assays for their ability to induce primary human T cells to kill human tumor cells in a tumor target dependent manner.

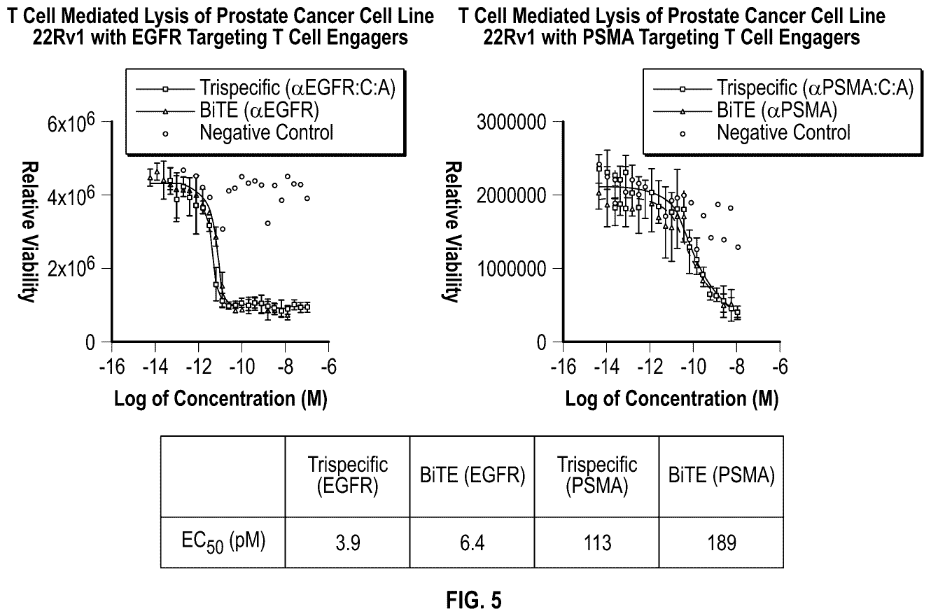

Trispecific molecules containing single domain antibody derived tumor targeting domains against EGFR or PSMA can induce potent cell killing in a manner comparable to bispecific T cell engagers (BiTE), see FIG. 5.

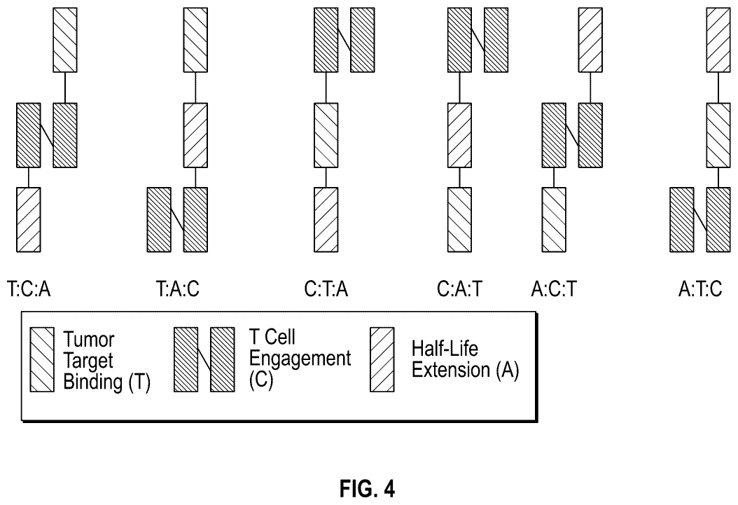

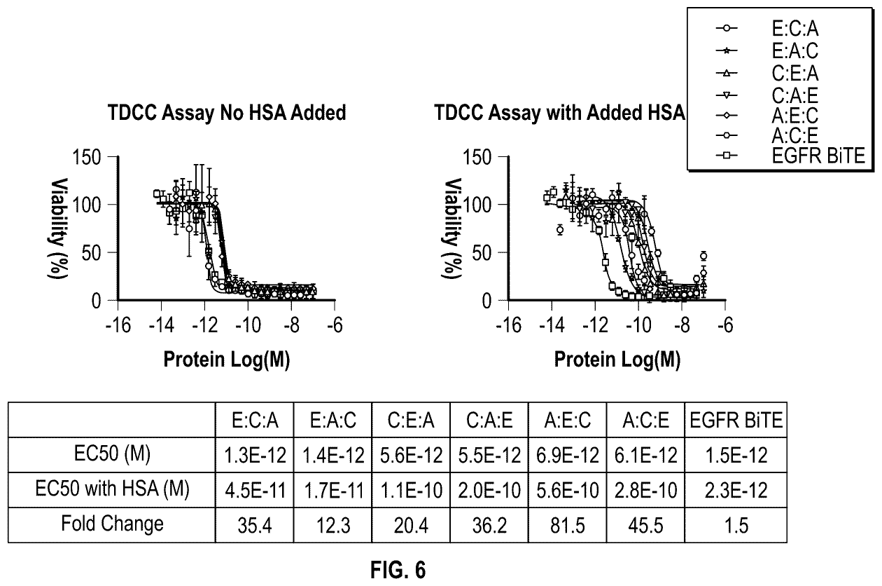

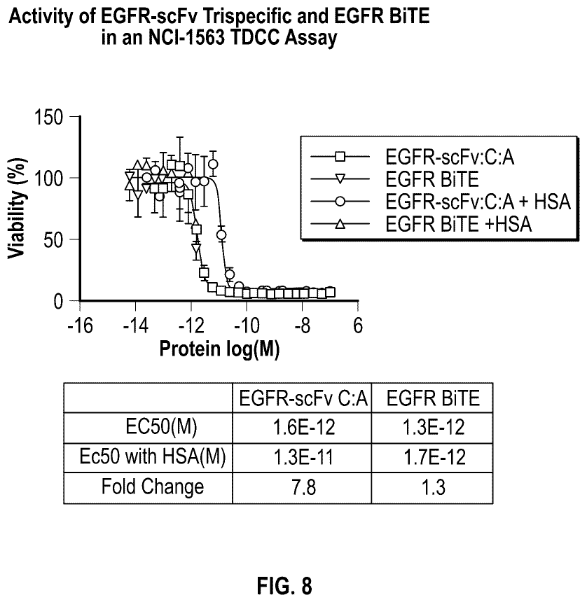

Six EGFR targeting trispecific molecules with a single domain anti-EGFR antibody (see FIG. 4) and a trispecific molecule containing an anti-EGFR scFv were tested in TDCC assays using NCI-1563 human lung adenocarcinoma cell line. For comparison, an EGFR BiTE was included in each assay (Lutterbuese et al. 2007. PNAS 107: 12605-12610). All 7 EFGR targeting trispecific molecule configurations were demonstrated to effectively kill target cells (see representative data in Tables 3 and 4 and FIGS. 6 and 8) with a similar potency to the EGFR BiTE. The TDCC assay was also performed with the addition of 15 mg/ml human serum albumin to assess the impact of albumin binding on the TDCC activity of the trispecific molecules. As expected, the potency of the EGFR BiTE, which lacks an albumin binding domain, was similar in the absence or presence of albumin. The potencies of the trispecific molecules decreased in the presence of albumin, but the amount of the decrease was dependent on the configuration of the molecule. The configurations whose potencies decreased the least in the presence of albumin were the EGFR-scFv:C:A and E:A:C (anti-EGFR-scFv:anti-CD3E-scFv:anti-ALB-sdAb and anti-EGFR-sdAb:anti-ALB-sdAb:anti-CD3E-scFv).

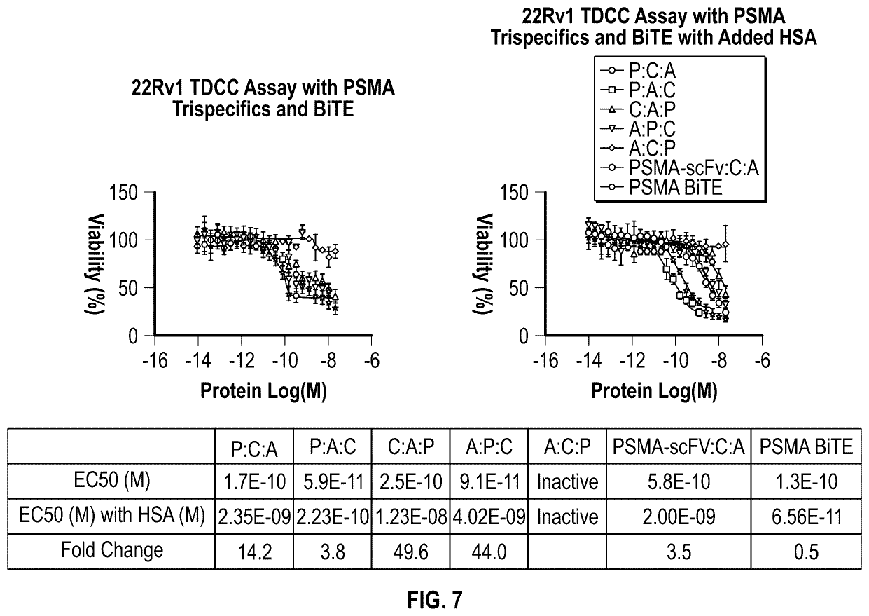

To demonstrate that the results of the EGFR targeting trispecific molecules may apply to all trispecific molecules, five PSMA targeting trispecific molecules with a single domain anti-PSMA antibody and a trispecific molecule containing an anti-PSMA scFv were tested in a TDCC assay using 22Rv1 human prostate carcinoma epithelial cell line. For comparison, a PSMA BiTE (pasotuxizumab) was included in the assay. Representative results are found in Table 5 and FIG. 7. Most of the PSMA targeting trispecific molecules had similar activity to the PSMA BiTE in the TDCC assay except for a trispecific molecule with a A:C:P configuration (anti-PSMA-sdAb:anti-CD3E-scFv:anti-ALB-sdAb). These trispecific molecules were also tested in a TDCC assay containing 15 mg/ml human serum albumin to assess the impact of albumin binding on the TDCC activity of the trispecific molecules. As expected, the potency of the PSMA BiTE, which lacks an albumin binding domain, was similar in the absence or presence of albumin. The potencies of the trispecific molecules decreased in the presence of albumin, but the amount of the decrease was dependent on the configuration of the molecule. The configurations whose potency decreased the least in the presence of albumin was the P:A:C (anti-PSMA-sdAb:anti-ALB-sdAb:anti-CD3E-scFv).

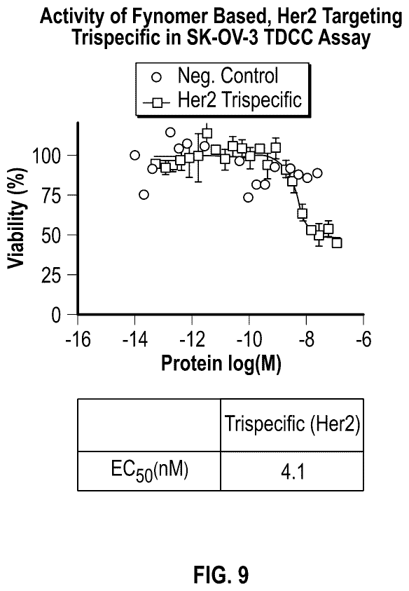

The trispecific molecules described here can utilize various modalities to target tumor cells. FIGS. 5, 6 and 7 show trispecific molecules with sdAb derived tumor targeting domains, and FIGS. 7 and 8 show that trispecific molecules with a scFv derived tumor binding domain can work equally well. FIG. 9 demonstrates that the tumor targeting domain is not limited to constructs derived from antibodies like sdAbs and scFvs, but that non-immunoglobulin domains can also work. In this example, a 7 kDa fynomer specific to Her2 is used to redirect resting human T cells to kill the human ovarian cancer cells.

### Example 11: Cytokine Production Assays with Trispecific Molecules

In order to show that the trispecific molecules tested here did activate T cells and redirected these T cells to kill tumor cells, the production of the cytokines TNFα and IFNγ was determined in parallel to the cell killing activity of the T cells, since T cells produce these cytokines as they get activated.

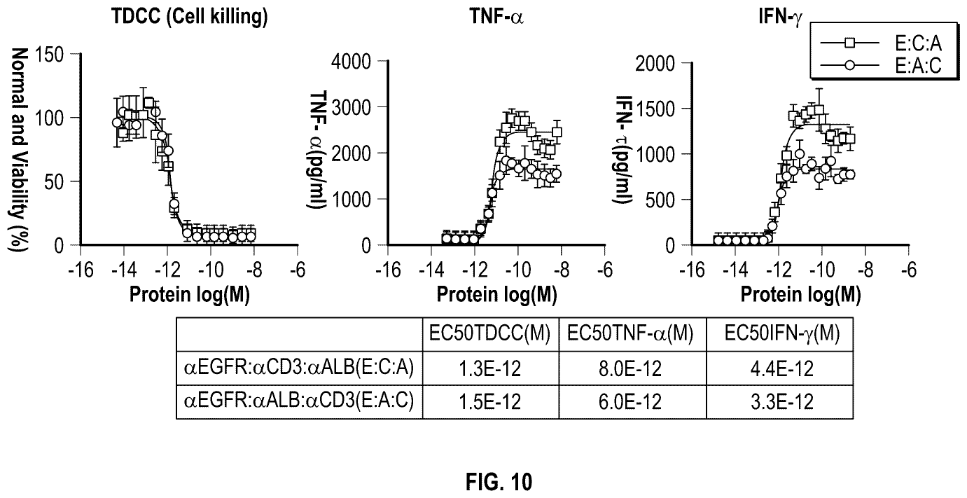

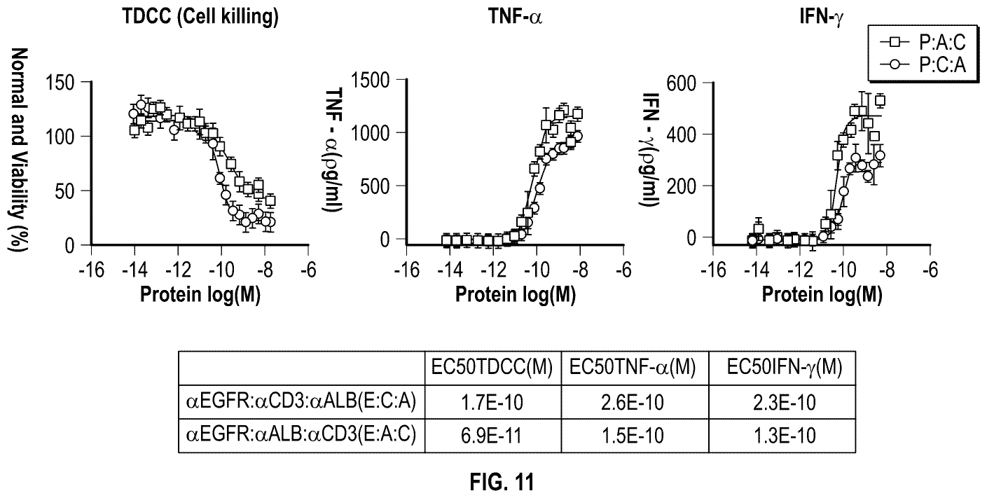

As shown in FIGS. 10 and 11, the four tested EGFR and PSMA targeting trispecific molecules stimulated TNFα and Interferon γ production with potency similar to their cell killing activity. These data are consistent with the statement that the trispecific molecules activate the T Cells when engaging target cells.

### Example 12: Diffusion Assays

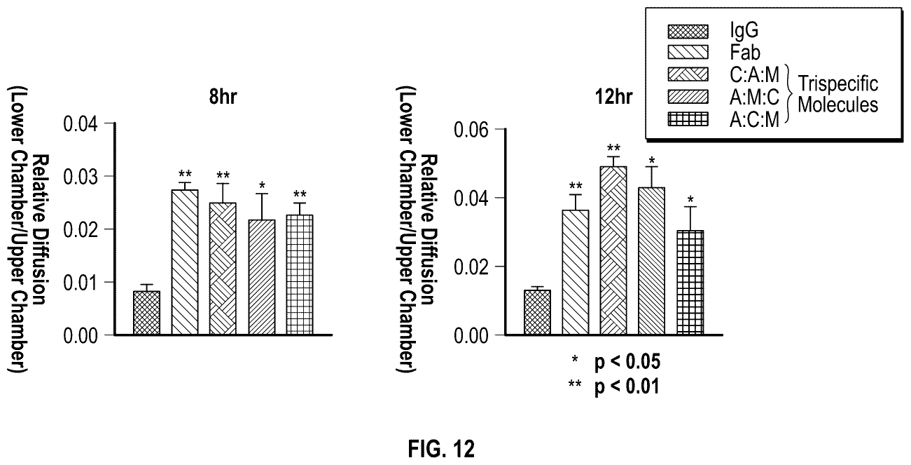

The trispecific molecules analyzed here are smaller than conventional IgG molecules, and hence are expected to diffuse faster and penetrate tissues better than monoclonal antibodies. A diffusion/migration assay through matrigel was developed to assess this property. For this purpose, transwell assay plates were coated with matrigel, a gelatinous protein mixture resembling the complex extracellular environment found in many tissues. Trispecific molecules, full length IgG or Fab fragments were added to the upper chamber. After eight and 12 hours, the lower chamber was assessed for the amount of macromolecule able to migrate through the matrix. As shown in FIG. 12, the trispecific molecules migrated at both time points at a rater much faster than full length IgG molecules.

### Example 13: Identification of Anti-CD3 scFv Variants with Varying Affinities for Human CD38

### Characterization of Parental Anti-CD3ε Phage

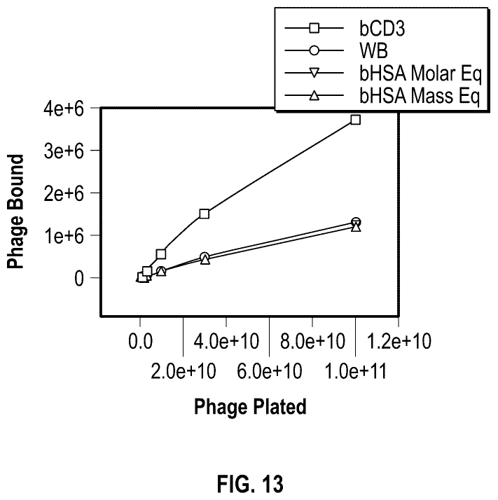

The parental anti-CD3ε showed good binding to biotin-CD3ε and low binding to biotin-HSA (FIG. 13).

Anti-CD3ε scFv Phage Libraries

A single substitution library was provided for the heavy chain CDR1, heavy chain CDR2, heavy chain CDR3, light chain CDR1, light chain CDR2, and light chain CDR3 domains. Residues were varied one at a time via mutagenesis.

### Selection of Clones and Determination of Binding Affinity

Single substitution libraries were bound to biotinylated hu-CD3ε, washed, eluted, and counted. Biotinylated cynoCD3 was used as the round1 selection target, and washed for 4 hours after combinatorial phage binding from the two independent libraries (˜2× selection). Biotinylated hu-CD3 was used as the round 2 selection target, and washed for 3 hours after binding of both libraries (<2× selection). PCRed inserts from the second round of selection were subcloned into the pcDNA3.4 His6 expression vector. 180 clones were picked and DNA was purified, sequenced, and transfected into Expi293. A panel of sixteen clones with a range of affinities for human CD3ε were selected for more precise K_{d} determination (Table 6).

Table 1 summarizes the affinities of trispecific molecules containing an EGFR targeting single domain antibody for the three target antigens. Key to table abbreviations: E=anti-EGFR single domain antibody, C=anti-CD3E scFv, A=anti-albumin single domain antibody.

| | Affinity | | |
|---|---|---|---|
| | huEGFR | huCD3 | HSA |
| Trispecific | K_{D} | K_{D} | K_{D} |
| Configuration | (nM) | (nM) | (nM) |
| E:C:A | 0.4 | 4.7 | 22.2 |
| E:A:C | 0.8 | 4.7 | 17.7 |
| C:E:A | 44.8 | 4.0 | 17.9 |
| C:A:E | 54.5 | 4.2 | 17.2 |
| A:E:C | 48.3 | 4.5 | 4.1 |
| A:C:E | 49.1 | 3.7 | 3.8 |

Table 2 summarizes the affinities of trispecific molecules containing a PSMA targeting single domain antibody for the three target antigens. Key to table abbreviations: P=anti-PSMA single domain antibody, C=anti-CD3E scFv, A=anti-albumin single domain antibody.

| | Affinity | | |
|---|---|---|---|
| | huPSMA | huCD3 | HSA |
| Trispecific | K_{D} | K_{D} | K_{D} |
| Configuration | (nM) | (nM) | (nM) |
| P:C:A | 16.7 | 3.6 | 24.0 |
| P:A:C | 31.6 | 4.1 | 21.0 |
| C:A:P | 51.0 | 4.2 | 21.7 |
| A:P:C | 25.0 | 2.1 | 3.5 |
| A:C:P | 39.7 | 2.7 | 3.5 |

Table 3 summarizes the potencies of trispecific molecules containing an EGFR targeting single domain antibody in cell killing assays. EC50 values are presented as molar concentrations. Key to table abbreviations: E=anti-EGFR single domain antibody, C=anti-CD3E scFv, A=anti-albumin single domain antibody.

| | EC50 | EC50 with | Fold |
|---|---|---|---|
| Protein | (M) | HSA (M) | change |
| E:C:A | 1.30E−12 | 4.50E−11 | 35.4 |
| E:A:C | 1.40E−12 | 1.70E−11 | 12.3 |
| C:E:A | 5.60E−12 | 1.10E−10 | 20.4 |
| C:A:E | 5.50E−12 | 2.00E−10 | 36.2 |
| A:E:C | 6.90E−12 | 5.60E−10 | 81.5 |
| A:C:E | 6.10E−12 | 2.80E−10 | 45.5 |
| EGFR BITE | 1.50E−12 | 2.30E−12 | 1.5 |

Table 4 summarizes the potencies of trispecific molecules containing an EGFR targeting scFv antibody and a BiTE molecule in cell killing assays. EC50 values are presented as molar concentrations. Key to table abbreviations: E=anti-EGFR single domain antibody, C=anti-CD3E scFv, A=anti-albumin single domain antibody.

| | EC50 | EC50 with | Fold |
|---|---|---|---|
| Protein | (M) | HSA (M) | change |
| EGFR-scFv:C:A | 1.60E−12 | 1.30E−13 | 7.8 |
| EGFR BITE | 1.30E−12 | 1.70E−12 | 1.3 |

Table 5 summarizes the potencies of trispecific molecules containing a PSMA targeting single domain antibody in cell killing assays. EC50 values are presented as molar concentrations. Key to table abbreviations: P=anti-PSMA single domain antibody, C=anti-CD3E scFv,A=anti-albumin single domain antibody.

| | EC50 | EC50 with | Fold |
|---|---|---|---|
| Protein | (M) | HSA (M) | change |
| P:C:A | 1.70E−10 | 2.35E−09 | 14.2 |
| P:A:C | 5.90E−11 | 2.23E−10 | 3.8 |
| C:A:P | 2.50E−10 | 1.23E−08 | 49.6 |
| A:P:C | 9.10E−11 | 4.02E−09 | 44 |
| A:C:P | inactive | inactive | |
| PSMA-scFV:C:A | 5.80E−10 | 2.00E−09 | 3.5 |
| PSMA BITE | 1.30E−10 | 6.56E−11 | 0.5 |

Table 6 summarizes binding affinities of CD3e scFv phage libraries.

| anti-CD3e | KD (nM) | | | KD (nM) | | | cyno/hum |
|---|---|---|---|---|---|---|---|
| scFv | hum CD3e | kon(1/Ms) | kdis(1/s) | cyno CD3e | kon(1/Ms) | kdis(1/s) | ratio |
| wt | 4.4 | 4.71E+05 | 2.07E−03 | 3.9 | 4.63E+05 | 1.83E−03 | 0.9 |
| 2B2 | 3.8 | 6.08E+05 | 2.32E−03 | 3.5 | 5.57E+05 | 1.93E−03 | 0.9 |
| 9F2 | 4.1 | 3.61E+05 | 1.33E−03 | 3.4 | 3.38E+05 | 1.05E−03 | 0.8 |
| 5A2 | 4.3 | 5.66E+05 | 2.36E−03 | 4.2 | 4.75E+05 | 1.93E−03 | 1.0 |
| 6A2 | 4.7 | 5.22E+05 | 2.48E−03 | 4.9 | 4.56E+05 | 2.22E−03 | 1.0 |
| 2D2 | 6.4 | 5.27E+05 | 3.38E−03 | 6.6 | 4.71E+05 | 3.09E−03 | 1.0 |
| 3F2 | 8.0 | 7.04E+05 | 5.02E−03 | 6.6 | 7.12E+05 | 4.38E−03 | 0.8 |
| 2E4 | 14.4 | 4.16E+05 | 5.99E−03 | 13.2 | 4.04E+05 | 5.32E−03 | 0.9 |
| 2H2 | 16.0 | 5.87E+05 | 9.06E−03 | 16.0 | 5.25E+05 | 8.37E−03 | 1.0 |
| 10B2 | 17.9 | 4.90E+05 | 8.74E−03 | 16.6 | 4.93E+05 | 8.15E−03 | 0.9 |
| 1A2 | 19.9 | 5.99E+05 | 1.19E−02 | 17 | 5.31E+05 | 9.03E−03 | 0.9 |
| 1C2 | 36.8 | 6.63E+05 | 2.44E−02 | 30 | 6.69E+05 | 1.97E−02 | 0.8 |
| 2A4 | 46.3 | 3.64E+05 | 1.66E−02 | 43.4 | 3.53E+05 | 1.53E−02 | 0.9 |
| 10E4 | 49.8 | 5.22E+05 | 2.60E−02 | 46.8 | 5.08E+05 | 2.38E−02 | 0.9 |
| 8A5 | 109 | 7.46E+05 | 8.10E−02 | 103 | 7.23E+05 | 7.44E−02 | 0.9 |
| 2G5 | 117 | 9.94E+05 | 1.15E−01 | 115 | 9.64E+05 | 1.11E−01 | 1.0 |
| 1G4 | 132.9 | 1.67E+05 | 2.20E−02 | 133.7 | 1.64E+05 | 2.19E−02 | 1.0 |

TABLE 7
Sequences

| SEQ ID | | | |
|---|---|---|---|
| NO: | Construct | Abbreviation | Sequence |
| 1 | αEGFR:αCD3: | E:C:A | EVQLVESGGGLVQAGGSLRLSCAASGRTFSSYAMGWFRQAPGKE |
| | αAlbumin | | REFVVAINWSSGSTYYADSVKGRFTISRDNAKNTMYLQMNSLKPE |
| | Trispecific | | DTAVYYCAAGYQINSGNYNFKDYEYDYWGQGTQVTVSSGGGGS |
| | | | GGGSEVQLVESGGGLVQPGGSLKLSCAASGFTFNKYAMNWVRQ |
| | | | APGKGLEWVARIRSKYNNYATYYADSVKDRFTISRDDSKNTAYLQ |
| | | | MNNLKTEDTAVYYCVRHGNFGNSYISYWAYWGQGTLVTVSSGG |
| | | | GGSGGGGSGGGGSQTVVTQEPSLTVSPGGTVTLTCGSSTGAVTS |
| | | | GNYPNWVQQKPGQAPRGLIGGTKFLAPGTPARFSGSLLGGKAAL |
| | | | TLSGVQPEDEAEYYCVLWYSNRWVFGGGTKLTVLGGGGSGGGS |
| | | | EVQLVESGGGLVQPGNSLRLSCAASGFTFSSFGMSWVRQAPGKG |
| | | | LEWVSSISGSGSDTLYADSVKGRFTISRDNAKTTLYLQMNSLRPED |
| | | | TAVYYCTIGGSLSRSSQGTLVTVSSHHHHHH |
| 2 | αEGFR: | E:A:C | EVQLVESGGGLVQAGGSLRLSCAASGRIFSSYAMGWFRQAPGKE |
| | αAlbumin: | | REFVVAINWSSGSTYYADSVKGRFTISRDNAKNTMYLQMNSLKPE |
| | αCD3 | | DTAVYYCAAGYQINSGNYNFKDYEYDYWGQGTQVTVSSGGGGS |
| | Trispecific | | GGGSEVQLVESGGGLVQPGNSLRLSCAASGFTFSSFGMSWVRQA |
| | | | PGKGLEWVSSISGSGSDTLYADSVKGRFTISRDNAKTTLYLQMNSL |
| | | | RPEDTAVYYCTIGGSLSRSSQGTLVTVSSGGGGSGGGSEVQLVES |
| | | | GGGLVQPGGSLKLSCAASGFTFNKYAMNWVRQAPGKGLEWVA |
| | | | RIRSKYNNYATYYADSVKDRFTISRDDSKNTAYLQMNNLKTEDTA |
| | | | VYYCVRHGNFGNSYISYWAYWGQGTLVTVSSGGGGSGGGGSGG |
| | | | GGSQTVVTQEPSLTVSPGGTVTLTCGSSTGAVTSGNYPNWVQQK |
| | | | PGQAPRGLIGGTKFLAPGTPARFSGSLLGGKAALTLSGVQPEDEAE |
| | | | YYCVLWYSNRWVFGGGTKLTVLHHHHHH |
| 3 | αCD3:αEGFR: | C:E:A | EVQLVESGGGLVQPGGSLKLSCAASGFTFNKYAMNWVRQAPGK |
| | αAlbumin | | GLEWVARIRSKYNNYATYYADSVKDRFTISRDDSKNTAYLQMNNL |
| | Trispecific | | KTEDTAVYYCVRHGNFGNSYISYWAYWGQGTLVTVSSGGGGSG |
| | | | GGGSGGGGSQTVVTQEPSLTVSPGGTVTLTCGSSTGAVTSGNYP |
| | | | NWVQQKPGQAPRGLIGGTKFLAPGTPARFSGSLLGGKAALTLSG |
| | | | VQPEDEAEYYCVLWYSNRWVFGGGTKLTVLGGGGSGGGSEVQL |
| | | | VESGGGLVQAGGSLRLSCAASGRTFSSYAMGWFRQAPGKEREFV |
| | | | VAINWSSGSTYYADSVKGRFTISRDNAKNTMYLQMNSLKPEDTA |
| | | | VYYCAAGYQINSGNYNFKDYEYDYWGQGTQVIVSSGGGGSGGG |
| | | | SEVQLVESGGGLVQPGNSLRLSCAASGFIFSSFGMSWVRQAPGK |
| | | | GLEWVSSISGSGSDTLYADSVKGRFTISRDNAKTTLYLQMNSLRPE |
| | | | DTAVYYCTIGGSLSRSSQGTLVTVSSHHHHHH* |
| 4 | αCD3: | C:A:E | EVQLVESGGGLVQPGGSLKLSCAASGFTFNKYAMNWVRQAPGK |
| | αAlbumin: | | GLEWVARIRSKYNNYATYYADSVKDRFTISRDDSKNTAYLQMNNL |
| | αEGFR | | KTEDTAVYYCVRHGNFGNSYISYWAYWGQGTLVTVSSGGGGSG |
| | Trispecific | | GGGSGGGGSQTVVTQEPSLTVSPGGTVTLTCGSSTGAVTSGNYP |
| | | | NWVQQKPGQAPRGLIGGTKFLAPGTPARFSGSLLGGKAALTLSG |
| | | | VQPEDEAEYYCVLWYSNRWVFGGGTKLTVLGGGGSGGGSEVQL |
| | | | VESGGGLVQPGNSLRLSCAASGFTFSSFGMSWVRQAPGKGLEW |
| | | | VSSISGSGSDTLYADSVKGRFTISRDNAKTTLYLQMNSLRPEDTAV |
| | | | YYCTIGGSLSRSSQGTLVTVSSGGGGSGGGSEVQLVESGGGLVQA |
| | | | GGSLRLSCAASGRTFSSYAMGWFRQAPGKEREFVVAINWSSGST |
| | | | YYADSVKGRFTISRDNAKNTMYLQMNSLKPEDTAVYYCAAGYQI |
| | | | NSGNYNFKDYEYDYWGQGTQVTVSSHHHHHH |
| 5 | αAlbumin: | A:E:C | EVQLVESGGGLVQPGNSLRLSCAASGFTFSSFGMSWVRQAPGKG |
| | αEGFR:αCD3 | | LEWVSSISGSGSDTLYADSVKGRFTISRDNAKTTLYLQMNSLRPED |
| | Trispecific | | TAVYYCTIGGSLSRSSQGTLVTVSSGGGGSGGGSEVQLVESGGGL |
| | | | VQAGGSLRLSCAASGRTFSSYAMGWFRQAPGKEREFVVAINWSS |
| | | | GSTYYADSVKGRFTISRDNAKNTMYLQMNSLKPEDTAVYYCAAG |
| | | | YQINSGNYNFKDYEYDYWGQGTQVTVSSGGGGSGGGSEVQLVE |
| | | | SGGGLVQPGGSLKLSCAASGFTFNKYAMNWVRQAPGKGLEWV |
| | | | ARIRSKYNNYATYYADSVKDRFTISRDDSKNTAYLQMNNLKTEDT |
| | | | AVYYCVRHGNFGNSYISYWAYWGQGTLVTVSSGGGGSGGGGSG |
| | | | GGGSQTVVTQEPSLTVSPGGTVTLTCGSSTGAVTSGNYPNWVQQ |
| | | | KPGQAPRGLIGGTKFLAPGTPARFSGSLLGGKAALTLSGVQPEDEA |
| | | | EYYCVLWYSNRWVFGGGTKLTVLHHHHHH* |
| 6 | αAlbumin: | A:C:E | EVQLVESGGGLVQPGNSLRLSCAASGFTFSSFGMSWVRQAPGKG |
| | αCD3:αEGFR | | LEWVSSISGSGSDTLYADSVKGRFTISRDNAKTTLYLQMNSLRPED |
| | Trispecific | | TAVYYCTIGGSLSRSSQGTLVTVSSGGGGSGGGSEVQLVESGGGL |
| | | | VQPGGSLKLSCAASGFTFNKYAMNWVRQAPGKGLEWVARIRSK |
| | | | YNNYATYYADSVKDRFTISRDDSKNTAYLQMNNLKTEDTAVYYCV |
| | | | RHGNFGNSYISYWAYWGQGTLVTVSSGGGGSGGGGSGGGGSQ |
| | | | TVVTQEPSLTVSPGGTVTLTCGSSTGAVTSGNYPNWVQQKPGQA |
| | | | PRGLIGGTKFLAPGTPARFSGSLLGGKAALTLSGVQPEDEAEYYCV |
| | | | LWYSNRWVFGGGTKLTVLGGGGSGGGSEVQLVESGGGLVQAG |
| | | | GSLRLSCAASGRTFSSYAMGWFRQAPGKEREFVVAINWSSGSTYY |
| | | | ADSVKGRFTISRDNAKNTMYLQMNSLKPEDTAVYYCAAGYQINS |
| | | | GNYNFKDYEYDYWGQGTQVTVSSHHHHHH* |
| 7 | EGFR BiTE | | DILLTQSPVILSVSPGERVSFSCRASQSIGTNIHWYQQRTNGSPRLL |
| | | | IKYASESISGIPSRFSGSGSGTDFTLSINSVESEDIADYYCQQNNNW |
| | | | PTTFGAGTKLELKGGGGSGGGGSGGGGSQVQLKQSGPGLVQPS |
| | | | QSLSITCTVSGFSLTNYGVHWVRQSPGKGLEWLGVIWSGGNTDY |
| | | | NTPFTSRLSINKDNSKSQVFFKMNSLQSNDTAIYYCARALTYYDYE |
| | | | FAYWGQGTLVTVSASGGGGSEVQLVESGGGLVQPGGSLKLSCAA |
| | | | SGFTFNKYAMNWVRQAPGKGLEWVARIRSKYNNYATYYADSVK |
| | | | DRFTISRDDSKNTAYLQMNNLKTEDTAVYYCVRHGNFGNSYISYW |
| | | | AYWGQGTLVTVSSGGGGSGGGGSGGGGSQTVVTQEPSLTVSPG |
| | | | GTVTLTCGSSTGAVTSGNYPNWVQQKPGQAPRGLIGGTKFLAPG |
| | | | TPARFSGSLLGGKAALTLSGVQPEDEAEYYCVLWYSNRWVFGGG |
| | | | TKLTVLHHHHHH |
| 8 | EGFR-scFv:C:A | EGFR- | DILLTQSPVILSVSPGERVSFSCRASQSIGTNIHWYQQRTNGSPRLL |
| | | scFv:C:A | IKYASESISGIPSRFSGSGSGTDFTLSINSVESEDIADYYCQQNNNW |
| | | | PTTFGAGTKLELKGGGGSGGGGSGGGGSQVQLKQSGPGLVQPS |
| | | | QSLSITCTVSGFSLTNYGVHWVRQSPGKGLEWLGVIWSGGNTDY |
| | | | NTPFTSRLSINKDNSKSQVFFKMNSLQSNDTAIYYCARALTYYDYE |
| | | | FAYWGQGTLVTVSASGGGGSEVQLVESGGGLVQPGGSLKLSCAA |
| | | | SGFTFNKYAMNWVRQAPGKGLEWVARIRSKYNNYATYYADSVK |
| | | | DRFTISRDDSKNTAYLQMNNLKTEDTAVYYCVRHGNFGNSYISYW |
| | | | AYWGQGTLVTVSSGGGGSGGGGSGGGGSQTVVTQEPSLTVSPG |
| | | | GTVTLTCGSSTGAVTSGNYPNWVQQKPGQAPRGLIGGTKFLAPG |
| | | | TPARFSGSLLGGKAALTLSGVQPEDEAEYYCVLWYSNRWVFGGG |
| | | | TKLTVLGGGGSGGGSEVQLVESGGGLVQPGNSLRLSCAASGFTFS |
| | | | SFGMSWVRQAPGKGLEWVSSISGSGSDTLYADSVKGRFTISRDN |
| | | | AKTTLYLQMNSLRPEDTAVYYCTIGGSLSRSSQGTLVTVSSHHHHH |
| | | | H |
| 9 | αPSMA:αCD3: | P:C:A | EVQLVESGGGLVQPGGSLTLSCAASRFMISEYSMHWVRQAPGKG |
| | αAlbumin | | LEWVSTINPAGTTDYAESVKGRFTISRDNAKNTLYLQMNSLKPED |
| | Trispecific | | TAVYYCDGYGYRGQGTQVTVSSGGGGSGGGSEVQLVESGGGLV |
| | | | QPGGSLKLSCAASGFTFNKYAMNWVRQAPGKGLEWVARIRSKY |
| | | | NNYATYYADSVKDRFTISRDDSKNTAYLQMNNLKTEDTAVYYCVR |
| | | | HGNFGNSYISYWAYWGQGTLVTVSSGGGGSGGGGSGGGGSQT |
| | | | VVTQEPSLTVSPGGTVTLTCGSSTGAVTSGNYPNWVQQKPGQAP |
| | | | RGLIGGTKFLAPGTPARFSGSLLGGKAALTLSGVQPEDEAEYYCVL |
| | | | WYSNRWVFGGGTKLTVLGGGGSGGGSEVQLVESGGGLVQPGN |
| | | | SLRLSCAASGFTFSSFGMSWVRQAPGKGLEWVSSISGSGSDTLYA |
| | | | DSVKGRFTISRDNAKTTLYLQMNSLRPEDTAVYYCTIGGSLSRSSQ |
| | | | GTLVTVSSHHHHHH |
| 10 | αPSMA: | P:A:C | EVQLVESGGGLVQPGGSLTLSCAASRFMISEYSMHWVRQAPGKG |
| | αAlbumin: | | LEWVSTINPAGTTDYAESVKGRFTISRDNAKNTLYLQMNSLKPED |
| | aCD3 | | TAVYYCDGYGYRGQGTQVIVSSGGGGSGGGSEVQLVESGGGLV |
| | Trispecific | | QPGNSLRLSCAASGFTFSSFGMSWVRQAPGKGLEWVSSISGSGS |
| | | | DTLYADSVKGRFTISRDNAKTTLYLQMNSLRPEDTAVYYCTIGGSL |
| | | | SRSSQGTLVTVSSGGGGSGGGSEVQLVESGGGLVQPGGSLKLSCA |
| | | | ASGFTFNKYAMNWVRQAPGKGLEWVARIRSKYNNYATYYADSV |
| | | | KDRFTISRDDSKNTAYLQMNNLKTEDTAVYYCVRHGNFGNSYISY |
| | | | WAYWGQGTLVTVSSGGGGSGGGGSGGGGSQTVVTQEPSLTVS |
| | | | PGGTVTLTCGSSTGAVTSGNYPNWVQQKPGQAPRGLIGGTKFLA |
| | | | PGTPARFSGSLLGGKAALTLSGVQPEDEAEYYCVLWYSNRWVFG |
| | | | GGTKLTVLHHHHHH |
| 11 | αCD3: | C:A:P | EVQLVESGGGLVQPGGSLKLSCAASGFTFNKYAMNWVRQAPGK |
| | αAlbumin: | | GLEWVARIRSKYNNYATYYADSVKDRFTISRDDSKNTAYLQMNNL |
| | αPSMA | | KTEDTAVYYCVRHGNFGNSYISYWAYWGQGTLVTVSSGGGGSG |
| | Trispecific | | GGGSGGGGSQTVVTQEPSLTVSPGGTVTLTCGSSTGAVTSGNYP |
| | | | NWVQQKPGQAPRGLIGGTKFLAPGTPARFSGSLLGGKAALTLSG |
| | | | VQPEDEAEYYCVLWYSNRWVFGGGTKLTVLGGGGSGGGSEVQL |
| | | | VESGGGLVQPGNSLRLSCAASGFTFSSFGMSWVRQAPGKGLEW |
| | | | VSSISGSGSDTLYADSVKGRFTISRDNAKTTLYLQMNSLRPEDTAV |
| | | | YYCTIGGSLSRSSQGTLVIVSSGGGGSGGGSEVQLVESGGGLVQP |
| | | | GGSLTLSCAASRFMISEYSMHWVRQAPGKGLEWVSTINPAGTTD |
| | | | YAESVKGRFTISRDNAKNTLYLQMNSLKPEDTAVYYCDGYGYRGQ |
| | | | GTQVTVSSHHHHHH |
| 12 | αAlbumin: | A:P:C | EVQLVESGGGLVQPGNSLRLSCAASGFTFSSFGMSWVRQAPGKG |
| | αPSMA:αCD3 | | LEWVSSISGSGSDTLYADSVKGRFTISRDNAKTTLYLQMNSLRPED |
| | Trispecific | | TAVYYCTIGGSLSRSSQGTLVTVSSGGGGSGGGSEVQLVESGGGL |
| | | | VQPGGSLTLSCAASRFMISEYSMHWVRQAPGKGLEWVSTINPAG |
| | | | TTDYAESVKGRFTISRDNAKNTLYLQMNSLKPEDTAVYYCDGYGY |
| | | | RGQGTQVIVSSGGGGSEVQLVESGGGLVQPGGSLKLSCAASGFT |
| | | | FNKYAMNWVRQAPGKGLEWVARIRSKYNNYATYYADSVKDRFTI |
| | | | SRDDSKNTAYLQMNNLKTEDTAVYYCVRHGNFGNSYISYWAYW |
| | | | GQGTLVTVSSGGGGSGGGGSGGGGSQTVVTQEPSLTVSPGGTV |
| | | | TLTCGSSTGAVTSGNYPNWVQQKPGQAPRGLIGGTKFLAPGTPA |
| | | | RFSGSLLGGKAALTLSGVQPEDEAEYYCVLWYSNRWVFGGGTKLT |
| | | | VLHHHHHH* |
| 13 | αAlbumin: | A:C:P | EVQLVESGGGLVQPGNSLRLSCAASGFTFSSFGMSWVRQAPGKG |
| | αCD3:αPSMA | | LEWVSSISGSGSDTLYADSVKGRFTISRDNAKTTLYLQMNSLRPED |
| | Trispecific | | TAVYYCTIGGSLSRSSQGTLVTVSSGGGGSGGGSEVQLVESGGGL |
| | | | VQPGGSLKLSCAASGFTFNKYAMNWVRQAPGKGLEWVARIRSK |
| | | | YNNYATYYADSVKDRFTISRDDSKNTAYLQMNNLKTEDTAVYYCV |
| | | | RHGNFGNSYISYWAYWGQGTLVTVSSGGGGSGGGGSGGGGSQ |
| | | | TVVTQEPSLTVSPGGTVTLTCGSSTGAVTSGNYPNWVQQKPGQA |
| | | | PRGLIGGTKFLAPGTPARFSGSLLGGKAALTLSGVQPEDEAEYYCV |
| | | | LWYSNRWVFGGGTKLTVLGGGGSGGGSEVQLVESGGGLVQPG |
| | | | GSLTLSCAASRFMISEYSMHWVRQAPGKGLEWVSTINPAGTTDY |
| | | | AESVKGRFTISRDNAKNTLYLQMNSLKPEDTAVYYCDGYGYRGQ |
| | | | GTQVTVSSHHHHHH* |
| 14 | αPSMA- | PSMA- | QVQLVESGGGLVKPGESLRLSCAASGFTFSDYYMYWVRQAPGKG |
| | scFv:αCD3: | scFv:C:A | LEWVAIISDGGYYTYYSDIIKGRFTISRDNAKNSLYLQMNSLKAEDT |
| | αAlbumin | | AVYYCARGFPLLRHGAMDYWGQGTLVTVSSGGGGSGGGGSGG |
| | Trispecific | | GGSDIQMTQSPSSLSASVGDRVTITCKASQNVDTNVAWYQQKP |
| | | | GQAPKSLIYSASYRYSDVPSRFSGSASGTDFTLTISSVQSEDFATYYC |
| | | | QQYDSYPYTFGGGTKLEIKSGGGGSEVQLVESGGGLVQPGGSLKL |
| | | | SCAASGFTFNKYAMNWVRQAPGKGLEWVARIRSKYNNYATYYA |
| | | | DSVKDRFTISRDDSKNTAYLQMNNLKTEDTAVYYCVRHGNFGNS |
| | | | YISYWAYWGQGTLVTVSSGGGGSGGGGSGGGGSQTVVTQEPSL |
| | | | TVSPGGTVTLTCGSSTGAVTSGNYPNWVQQKPGQAPRGLIGGTK |
| | | | FLAPGTPARFSGSLLGGKAALTLSGVQPEDEAEYYCVLWYSNRWV |
| | | | FGGGTKLTVLGGGGSGGGSEVQLVESGGGLVQPGNSLRLSCAAS |
| | | | GFTFSSFGMSWVRQAPGKGLEWVSSISGSGSDTLYADSVKGRFTI |
| | | | SRDNAKTTLYLQMNSLRPEDTAVYYCTIGGSLSRSSQGTLVTVSSH |
| | | | HHHHH |
| 15 | PSMA BiTE | | QVQLVESGGGLVKPGESLRLSCAASGFTFSDYYMYWVRQAPGKG |
| | | | LEWVAIISDGGYYTYYSDIIKGRFTISRDNAKNSLYLQMNSLKAEDT |
| | | | AVYYCARGFPLLRHGAMDYWGQGTLVTVSSGGGGSGGGGSGG |
| | | | GGSDIQMTQSPSSLSASVGDRVTITCKASQNVDTNVAWYQQKP |
| | | | GQAPKSLIYSASYRYSDVPSRFSGSASGTDFTLTISSVQSEDFATYYC |
| | | | QQYDSYPYTFGGGTKLEIKSGGGGSEVQLVESGGGLVQPGGSLKL |
| | | | SCAASGFTFNKYAMNWVRQAPGKGLEWVARIRSKYNNYATYYA |
| | | | DSVKDRFTISRDDSKNTAYLQMNNLKTEDTAVYYCVRHGNFGNS |
| | | | YISYWAYWGQGTLVTVSSGGGGSGGGGSGGGGSQTVVTQEPSL |
| | | | TVSPGGTVTLTCGSSTGAVTSGNYPNWVQQKPGQAPRGLIGGTK |
| | | | FLAPGTPARFSGSLLGGKAALTLSGVQPEDEAEYYCVLWYSNRWV |
| | | | FGGGTKLTVLHHHHHH |
| 16 | Her2- | | GVTLFVALYDYTSYNTRDLSFHKGEKFQILRMEDGVWWEARSLTT |
| | Fynomer:αCD3: | | GETGYIPSNYVAPVDSIQGGGGSGGGSEVQLVESGGGLVQPGGS |
| | αAlbumin | | LKLSCAASGFTFNKYAMNWVRQAPGKGLEWVARIRSKYNNYATY |
| | Trispecific | | YADSVKDRFTISRDDSKNTAYLQMNNLKTEDTAVYYCVRHGNFG |
| | | | NSYISYWAYWGQGTLVTVSSGGGGSGGGGSGGGGSQTVVTQEP |
| | | | SLTVSPGGTVTLTCGSSTGAVTSGNYPNWVQQKPGQAPRGLIGG |
| | | | TKFLAPGTPARFSGSLLGGKAALTLSGVQPEDEAEYYCVLWYSNR |
| | | | WVFGGGTKLTVLGGGGSGGGSEVQLVESGGGLVQPGNSLRLSC |
| | | | AASGFTFSSFGMSWVRQAPGKGLEWVSSISGSGSDTLYADSVKG |
| | | | RFTISRDNAKTTLYLQMNSLRPEDTAVYYCTIGGSLSRSSQGTLVTV |
| | | | SSHHHHHH* |
| 17 | αCD3: | C:A:M | EVQLVESGGGLVQPGGSLKLSCAASGFTFNKYAMNWVRQAPGK |
| | αAlbumin: | | GLEWVARIRSKYNNYATYYADSVKDRFTISRDDSKNTAYLQMNNL |
| | αMSLN | | KTEDTAVYYCVRHGNFGNSYISYWAYWGQGTLVTVSSGGGGSG |
| | Trispecific | | GGGSGGGGSQTVVTQEPSLTVSPGGTVTLTCGSSTGAVTSGNYP |
| | | | NWVQQKPGQAPRGLIGGTKFLAPGTPARFSGSLLGGKAALTLSG |
| | | | VQPEDEAEYYCVLWYSNRWVFGGGTKLTVLGGGGSGGGSEVQL |
| | | | VESGGGLVQPGNSLRLSCAASGFTFSSFGMSWVRQAPGKGLEW |
| | | | VSSISGSGSDTLYADSVKGRFTISRDNAKTTLYLQMNSLRPEDTAV |
| | | | YYCTIGGSLSRSSQGTLVTVSSGGGGSGGGSQVQLVQSGGGLVQ |
| | | | PGGSLRLSCAASDFDFAAYDMSWVRQAPGQGLEWVAIISHDGID |
| | | | KYYDDSVKGRFTISRDNSKNTLYLQMNTLRAEDTATYQCLRLGAV |
| | | | GQGTLVTVSSHHHHHH |
| 18 | αAlbumin: | A:M:C | EVQLVESGGGLVQPGNSLRLSCAASGFTFSSFGMSWVRQAPGKG |
| | αMSLN:αCD3 | | LEWVSSISGSGSDTLYADSVKGRFTISRDNAKTTLYLQMNSLRPED |
| | Trispecific | | TAVYYCTIGGSLSRSSQGTLVTVSSGGGGSGGGSQVQLVQSGGGL |
| | | | VQPGGSLRLSCAASDFDFAAYDMSWVRQAPGQGLEWVAIISHD |
| | | | GIDKYYDDSVKGRFTISRDNSKNTLYLQMNTLRAEDTATYQCLRLG |
| | | | AVGQGTLVTVSSGGGGSGGGSEVQLVESGGGLVQPGGSLKLSCA |
| | | | ASGFTFNKYAMNWVRQAPGKGLEWVARIRSKYNNYATYYADSV |
| | | | KDRFTISRDDSKNTAYLQMNNLKTEDTAVYYCVRHGNFGNSYISY |
| | | | WAYWGQGTLVTVSSGGGGSGGGGSGGGGSQTVVTQEPSLTVS |
| | | | PGGTVTLTCGSSTGAVTSGNYPNWVQQKPGQAPRGLIGGTKFLA |
| | | | PGTPARFSGSLLGGKAALTLSGVQPEDEAEYYCVLWYSNRWVFG |
| | | | GGTKLTVLHHHHHH |
| 19 | αAlbumin: | A:C:M | EVQLVESGGGLVQPGNSLRLSCAASGFTFSSFGMSWVRQAPGKG |
| | αCD3:αMSLN | | LEWVSSISGSGSDTLYADSVKGRFTISRDNAKTTLYLQMNSLRPED |
| | Trispecific | | TAVYYCTIGGSLSRSSQGTLVTVSSGGGGSGGGSEVQLVESGGGL |
| | | | VQPGGSLKLSCAASGFTFNKYAMNWVRQAPGKGLEWVARIRSK |
| | | | YNNYATYYADSVKDRFTISRDDSKNTAYLQMNNLKTEDTAVYYCV |
| | | | RHGNFGNSYISYWAYWGQGTLVTVSSGGGGSGGGGSGGGGSQ |
| | | | TVVTQEPSLTVSPGGTVTLTCGSSTGAVTSGNYPNWVQQKPGQA |
| | | | PRGLIGGTKFLAPGTPARFSGSLLGGKAALTLSGVQPEDEAEYYCV |
| | | | LWYSNRWVFGGGTKLTVLGGGGSGGGSQVQLVQSGGGLVQPG |
| | | | GSLRLSCAASDFDFAAYDMSWVRQAPGQGLEWVAIISHDGIDKY |
| | | | YDDSVKGRFTISRDNSKNTLYLQMNTLRAEDTATYQCLRLGAVGQ |
| | | | GTLVTVSSHHHHHH* |

TABLE 8
Sequences

| SEQ ID | | | | |
|---|---|---|---|---|
| NO: | Binder | Name | Chain | Sequence |
| 20 | CD3 | Anti- | | DIKLQQSGAELARPGASVKMSCKTSGYTFTRYTMHWVKQRPGQ |
| | | huCD3E- | | GLEWIGYINPSRGYTNYNQKFKDKATLTTDKSSSTAYMQLSSLTSE |
| | | scFv | | DSAVYYCARYYDDHYCLDYWGQGTTLTVSSVEGGSGGSGGSGGS |
| | | | | GGVDDIQLTQSPAIMSASPGEKVTMTCRASSSVSYMNWYQQKS |
| | | | | GTSPKRWIYDTSKVASGVPYRFSGSGSGTSYSLTISSMEAEDAATY |
| | | | | YCQQWSSNPLTFGAGTKLELK |
| 21 | CD3 | Anti- | Heavy | QVQLVESGGGVVQPGRSLRLSCAASGFKFSGYGMHWVRQAPGK |
| | | huCD3E | variable | GLEWVAVIWYDGSKKYYVDSVKGRFTISRDNSKNTLYLQMNSLR |
| | | | | AEDTAVYYCARQMGYWHFDLWGRGTLVTVSS |
| 22 | CD3 | Anti- | Light | EIVLTQSPATLSLSPGERATLSCRASQSVSSYLAWYQQKPGQAPRL |
| | | huCD3E | variable | LIYDASNRATGIPARFSGSGSGTDFTLTISSLEPEDFAVYYCQQRSN |
| | | | | WPPLTFGGGTKVEIK |
| 23 | CD3 | Anti- | Heavy | EVQLLESGGGLVQPGGSLRLSCAASGFTFSSFPMAWVRQAPGKG |
| | | huCD3E | variable | LEWVSTISTSGGRTYYRDSVKGRFTISRDNSKNTLYLQMNSLRAED |
| | | | | TAVYYCAKFRQYSGGFDYWGQGTLVTVSS |
| 24 | CD3 | Anti- | Light | DIQLTQPNSVSTSLGSTVKLSCTLSSGNIENNYVHWYQLYEGRSPT |
| | | huCD3E | variable | TMIYDDDKRPDGVPDRFSGSIDRSSNSAFLTIHNVAIEDEAIYFCHS |
| | | | | YVSSFNVFGGGTKLTVLR |
| 25 | CD3 | Anti- | | DVQLVQSGAEVKKPGASVKVSCKASGYTFTRYTMHWVRQAPGQ |
| | | huCD3E- | | GLEWIGYINPSRGYTNYADSVKGRFTITTDKSTSTAYMELSSLRSED |
| | | scFv | | TATYYCARYYDDHYCLDYWGQGTTVTVSSGEGTSTGSGGSGGSG |
| | | | | GADDIVLTQSPATLSLSPGERATLSCRASQSVSYMNWYQQKPGK |
| | | | | APKRWIYDTSKVASGVPARFSGSGSGTDYSLTINSLEAEDAATYYC |
| | | | | QQWSSNPLTFGGGTKVEIK |
| 26 | CD3 | Anti- | Heavy | QVQLVQSGGGVVQPGRSLRLSCKASGYTFTRYTMHWVRQAPGK |
| | | huCD3E | variable | GLEWIGYINPSRGYTNYNQKVKDRFTISRDNSKNTAFLQMDSLRP |
| | | (humanized | | EDTGVYFCARYYDDHYCLDYWGQGTPVTVSS |
| | | OKT3) | | |
| 27 | CD3 | Anti- | Light | DIQMTQSPSSLSASVGDRVTITCSASSSVSYMNWYQQTPGKAPK |
| | | huCD3E | variable | RWIYDTSKLASGVPSRFSGSGSGTDYTFTISSLQPEDIATYYCQQW |
| | | (humanized | | SSNPFTFGQGTKLQITR |
| | | OKT3) | | |
| 28 | CD3 | CD3 binder | Heavy | EVQLLESGGGLVQPGGSLRLSCAASGFTFSTYAMNWVRQAPGKG |
| | | | variable | LEWVSRIRSKYNNYATYYADSVKGRFTISRDDSKNTLYLQMNSLRA |
| | | | | EDTAVYYCVRHGNFGNSYVSWFAYWGQGTLVTVSS |
| 29 | CD3 | CD3 binder | Light | QAVVTQEPSLTVSPGGTVTLTCGSSTGAVTTSNYANWVQEKPGQ |
| | | | variable | AFRGLIGGTNKRAPGTPARFSGSLLGGKAALTLSGAQPEDEAEYYC |
| | | | | ALWYSNLWVFGGGTKLTVL |
| 30 | CD3 | CD3 binder | Heavy | EVQLVESGGGLVQPGRSLRLSCAASGFTFDDYTMHWVRQAPGK |
| | | | variable | GLEWVSGISWNSGSIGYADSVKGRFTISRDNAKKSLYLQMNSLRA |
| | | | | EDTALYYCAKDNSGYGHYYYGMDVWGQGTTVTVAS |
| 31 | CD3 | CD3 binder | Light | AEIVMTQSPATLSVSPGERATLSCRASQSVSSNLAWYQQKPGQA |
| | | | variable | PRLLIYGASTRATGIPARFSGSGSGTEFTLTISSLQSEDFAVYYCQHYI |
| | | | | NWPLTFGGGTKVEIK |
| 32 | CD3 | CD3 binder | Heavy | QVQLQQSGAELARPGASVKMSCKASGYTFTRSTMHWVKQRPG |
| | | | variable | QGLEWIGYINPSSAYTNYNQKFKDKATLTADKSSSTAYMQLSSLTS |
| | | | | EDSAVYYCASRQVHYDYNGFPYWGQGTLVTVSS |
| 33 | CD3 | CD3 binder | Light | QVVLTQSPAIMSAFPGEKVTMTCSASSSVSYMNWYQQKSGTSPK |
| | | | variable | RWIYDSSKLASGVPARFSGSGSGTSYSLTISSMETEDAATYYCQQ |
| | | | | WSRNPPTFGGGTKLQITR |
| 34 | CD3 | CD3 binder | Heavy | EVKLLESGGGLVQPKGSLKLSCAASGFTFNTYAMNWVRQAPGKG |
| | | | variable | LEWVARIRSKYNNYATYYADSVKDRFTISRDDSQSILYLQMNNLKT |
| | | | | EDTAMYYCVRHGNFGNSYVSWFAYWGQGTLVTVSA |
| 35 | CD3 | CD3 binder | Light | QAVVTQESALTTSPGETVTLTCRSSTGAVTTSNYANWVQEKPDHL |
| | | | variable | FTGLIGGTNKRAPGVPARFSGSLIGDKAALTITGAQTEDEAIYFCAL |
| | | | | WYSNLWVFGGGTKLTVLG |
| 36 | CD3 | humaninzed | | EVQLVESGGGLVQPGGSLRLSCAASGFTFNTYAMNWVRQAPGK |
| | | scFv | | GLEWVGRIRSKYNNYATYYADSVKGRFTISRDDSKNTLYLQMNSL |
| | | | | RAEDTAVYYCVRHGNFGNSYVSWFAYWGQGTLVTVSSGGGGSG |
| | | | | GGGSGGGGSQAVVTQEPSLTVSPGGTVTLTCGSSTGAVTTSNYA |
| | | | | NWVQQKPGQAPRGLIGGTNKRAPGVPARFSGSLLGGKAALTLSG |
| | | | | AQPEDEAEYYCALWYSNLWVFGGGTKLTVL |
| 37 | CD3 | CD3 binder | | QVQLQQSGAELARPGASVKMSCKASGYTFTRYTMHWVKQRPG |
| | | | | QGLEWIGYINPSRGYTNYNQKFKDKATLTTDKSSSTAYMQLSSLTS |
| | | | | EDSAVYYCARYYDDHYSLDYWGQGTTLTVSSAKTTPDIVLTQSPAI |
| | | | | MSASPGEKVTMTCSASSSVSYMNWYQQKSGTSPKRWIYDTSKLA |
| | | | | SGVPAHFRGSGSGTSYSLTISGMEAEDAATYYCQQWSSNPFTFGS |
| | | | | GTKLEINRADTAAAGSHHHHHH |
| 38 | HSA | VH only | | EVQLLESGGGLVQPGGSLRLSCAASGFTFSKYWMSWVRQAPGK |
| | | domain | | GLEWVSSIDFMGPHTYYADSVKGRFTISRDNSKNTLYLQMNSLRA |
| | | | | EDTAVYYCAKGRTSMLPMKGKFDYWGQGTLVTVSS |
| 39 | HSA | VH only | | EVQLLESGGGLVQPGGSLRLSCTASGFTFDEYNMSWVRQAPGKG |
| | | domain | | LEWVSTILPHGDRTYYADSVKGRFTISRDNSKNTLYLQMNSLRAE |
| | | | | DTAVYYCAKQDPLYRFDYWGQGTLVTVSS |
| 40 | HSA | VL only | | DIQMTQSPSSLSASVGDRVTITCRASQKIATYLNWYQQKPGKAPK |
| | | domain | | LLIYRSSSLQSAVPSRFSGSGSGTVFTLTISSLQPEDFATYYCQQTYA |
| | | | | VPPTFGQGTKVEIKR |
| 41 | HSA | VL only | | DIQMTQSPSSLSASVGDRVTITCRASQSISSYLNWYQQKPGKAPKL |
| | | domain | | LIYRNSPLQSGVPSRFSGSGSGTDFTLTISSLQPEDFATYYCQQTYR |
| | | | | VPPTFGQGTKVEIKR |
| 42 | HSA | MSA21 | | QVQLQESGGGLVQPGGSLRLSCEASGFTFSRFGMTWVRQAPGK |
| | | | | GVEWVSGISSLGDSTLYADSVKGRFTISRDNAKNTLYLQMNSLKPE |
| | | | | DTAVYYCTIGGSLNPGGQGTQVTVSS |
| 43 | HSA | NON- | | LKEAKEKAIEELKKAGITSDYYFDLINKAKTVEGVNALKDEILKA |
| | | NATURAL | | |
| | | CONSENSUS | | |
| | | ALBUMIN | | |
| | | BINDING | | |
| | | DOMAINS | | |
| 44 | HSA | anti-ALB | Heavy | EVQLLESGGGLVQPGGSLRLSCAVSGIDLSNYAINWVRQAPGKCL |
| | | FAB | variable | EWIGIIWASGTTFYATWAKGRFTISRDNSKNTVYLQMNSLRAEDT |
| | | | | AVYYCARTVPGYSTAPYFDLWGQGTLVTVSS |
| 45 | HSA | anti-ALB | Light | DIQMTQSPSSVSASVGDRVTITCQSSPSVWSNFLSWYQQKPGKA |
| | | FAB | variable | PKLLIYEASKLTSGVPSRFSGSGSGTDFTLTISSLQPEDFATYYCGGG |
| | | | | YSSISDTTFGCGTKVEIKRT |
| 46 | HSA | HSA VH only | | AVQLVESGGGLVQPGNSLRLSCAASGFTFRSFGMSWVRQAPGKE |
| | | | | PEWVSSISGSGSDTLYADSVKGRFTISRDNAKTTLYLQMNSLKPED |
| | | | | TAVYYCTIGGSLSRSSQGTQVTVSS |
| 47 | HSA | HSA VH only | | EVQLVESGGGLVQPGNSLRLSCAASGFTFSSFGMSWVRQAPGKG |
| | | | | LEWVSSISGSGSDTLYADSVKGRFTISRDNAKTTLYLQMNSLRPED |
| | | | | TAVYYCTIGGSLSRSSQGTLVTVSS |
| 48 | HSA | HSA VH only | | AVQLVESGGGLVQGGGSLRLACAASERIFDLNLMGWYRQGPGN |
| | | | | ERELVATCITVGDSTNYADSVKGRFTISMDYTKQTVYLHMNSLRPE |
| | | | | DTGLYYCKIRRTWHSELWGQGTQVTVSS |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A trispecific antigen-binding protein, wherein said protein comprises
(a) a first domain (A) which comprises a single chain variable fragment (scFv) that specifically binds to human CD3;
(b) a second domain (B) which comprises a single domain antibody (sdAb) that binds human serum albumin, wherein the second domain comprises an amino acid sequence of SEQ ID NO: 47; and
(c) a third domain (C) which comprises a scFv or sdAb that specifically binds to a target tumor antigen;
wherein the domains are linked in the order H₂N-(C)-(B)-(A)-COOH or by linkers L1 and L2 in the order H₂N-(C)-L1-(B)-L2-(A)-COOH.

2. The trispecific antigen-binding protein of claim 1, wherein the first domain is humanized or human.

3. The trispecific antigen-binding protein of claim 1, wherein linkers L1 and L2 are each, independently, (GS)ₙ (SEQ ID NO: 49), (GGS)ₙ (SEQ ID NO: 50), (GGGS)ₙ (SEQ ID NO: 51), (GGSG)ₙ (SEQ ID NO: 52), (GGSGG)ₙ (SEQ ID NO: 53), or (GGGGS)ₙ (SEQ ID NO: 54), wherein n is 1, 2, 3, 4, 5, 6, 7, 8, 9, or 10.

4. The trispecific antigen-binding protein of claim 1, wherein linkers L1 and L2 are each, independently, (GGGGS)₄ (SEQ ID NO: 55) or (GGGGS)₃ (SEQ ID NO: 56).

5. The trispecific antigen-binding protein of claim 1, wherein the first domain is specific for CD3ε (epsilon).

6. The trispecific antigen-binding protein of claim 1, wherein the first domain has crossreactivity with cynomolgus CD3.

7. A polynucleotide encoding the polypeptide of the trispecific antigen-binding protein of claim 1.

8. A vector comprising the polynucleotide of claim 7.

9. A pharmaceutical composition comprising (i) the trispecific antigen-binding protein according to claim 1 and (ii) a pharmaceutically acceptable carrier.

10. A pharmaceutical composition comprising (i) the vector according to claim 8 and (ii) a pharmaceutically acceptable carrier.

11. A trispecific antigen-binding protein, wherein said protein comprises
(a) a first domain (A) which comprises a single chain variable fragment (scFv) that specifically binds to human CD3;
(b) a second domain (B) which comprises a single domain antibody (sdAb) that binds human serum albumin, wherein the second domain comprises an amino acid sequence with a 90% identity to SEQ ID NO: 47; and
(c) a third domain (C) which comprises a scFv or sdAb that specifically binds to a target tumor antigen;
wherein the domains are linked in the order HN-(C)-(B)-(A)-COOH or by linkers L1 and L2.

12. The trispecific antigen-binding protein of claim 1, wherein the second domain comprises an amino acid sequence with a 95% identity to SEQ ID NO: 47.

13. The trispecific antigen-binding protein of claim 11, wherein the first domain is humanized or human.

14. The trispecific antigen-binding protein of claim 11, wherein linkers L1 and L2 are each, independently, (GS)ₙ (SEQ ID NO: 49), (GGS)ₙ (SEQ ID NO: 50), (GGGS)ₙ (SEQ ID NO: 51), (GGSG)ₙ (SEQ ID NO: 52), (GGSGG)ₙ (SEQ ID NO: 53), or (GGGGS)ₙ (SEQ ID NO: 54), wherein n is 1, 2, 3, 4, 5, 6, 7, 8, 9, or 10.

15. The trispecific antigen-binding protein of claim 11, wherein linkers L1 and L2 are each, independently, (GGGGS)₄ (SEQ ID NO: 55) or (GGGGS)₃ (SEQ ID NO: 56).

16. The trispecific antigen-binding protein of claim 11, wherein the first domain is specific for CD3ε (epsilon).

17. The trispecific antigen-binding protein of claim 11, wherein the first domain has crossreactivity with cynomolgus CD3.

18. A polynucleotide encoding the polypeptide of the trispecific antigen-binding protein of claim 11.

19. A vector comprising the polynucleotide of claim 18.

20. A pharmaceutical composition comprising (i) the trispecific antigen-binding protein according to claim 11 and (ii) a pharmaceutically acceptable carrier.

21. A pharmaceutical composition comprising (i) the vector according to claim 19 and (ii) a pharmaceutically acceptable carrier.
